(19) United States
(12) Patent Application Publication
(10) Pub. No.: US 2019/0300851 A1
(43) Pub. Date: Oct. 3, 2019

(54) HUMAN STEM CELL DERIVED ENDOTHELIAL CELLS, ENDOTHELIAL-HEPATOCYTE CO-CULTURE SYSTEM AND USES THEREOF

(71) Applicant: AGENCY FOR SCIENCE, TECHNOLOGY AND RESEARCH, Singapore (SG)

(72) Inventors: Christine CHEUNG, SINGAPORE (SG); Balakrishnan Chakrapani NARMADA, Singapore (SG); Yeek Teck GOH, Singapore (SG)

(21) Appl. No.: 16/302,615

(22) PCT Filed: May 16, 2017
(86) PCT No.: PCT/SG2017/050255

§ 371 Date: Nov. 16, 2018

(30) Foreign Application Priority Data
May 17, 2016 (SG) 10201603939U

(51) Int. Cl. C12N 5/071; G01N 33/50

(52) U.S. Cl. CPC C12N 5/069; C12N 5/067; G01N 33/5005; C12N 2501/115; C12N 2501/15; C12N 2503/02; C12N 2502/14; C12N 2506/02; C12N 2506/45; G01N 2500/10; C12N 2501/165

(57) ABSTRACT

The present disclosure provides a method of deriving endothelial cells, comprising (a) culturing lateral plate mesoderm cells under oxygen-deprived condition to obtain endothelial lineage cells; and (b) culturing endothelial cells from (a) on an extracellular matrix to maintain and expand the endothelial lineage cells. Also disclosed herein is a cell co-culture system comprising an endothelial cell culture and a hepatocyte cell culture, as well as a microfluidic-based system comprising said cell co-culture system. Also disclosed herein is a method of disease modelling or drug testing using said cell co-culture system or said microfluidic-based system.

### CROSS-REFERENCE TO RELATED APPLICATIONS

This application claims the benefit of priority of the Singapore provisional application No. 10201603939U, filed on 17 May 2016, the contents of it being hereby incorporated by reference in its entirety for all purposes.

### FIELD OF THE INVENTION

The present invention relates to cell biology and biochemistry, in particular methods of deriving and maintaining cells, in particular endothelial cells. The present invention also relates to a cell co-culture system and uses thereof.

### BACKGROUND OF THE INVENTION

Cardiovascular diseases are a group of disorders of the heart and blood vessels and they include: coronary heart disease (disease of the blood vessels supplying the heart muscle); cerebrovascular disease (disease of the blood vessels supplying the brain); peripheral arterial disease (disease of blood vessels supplying the arms and legs); rheumatic heart disease-(damage to the heart muscle and heart valves from rheumatic fever, caused by streptococcal bacteria); congenital heart disease (malformations of heart structure existing at birth); deep vein thrombosis and pulmonary embolism (blood clots in the leg veins, which can dislodge and move to the heart and lungs). Cardiovascular diseases are the leading cause of death globally: more people die annually from cardiovascular diseases than from any other cause. An estimated 17.5 million people died from cardiovascular diseases in 2012, representing 31% of all global deaths. Of these deaths, an estimated 7.4 million were due to coronary heart disease and 6.7 million were due to stroke.

There is an increasing need for developing novel drugs and pharmaceutical substances for the prevention and treatment of cardiovascular diseases. Currently existing preclinical models can provide a critical window into human physiology for determining drug safety and efficacy. However, these models typically involve exhaustive in vivo animal testing, while many animal models often fail to capture human-specific features, and offer only limited control of and insight into specific mechanisms. For example, existing heart models of cardio-toxicity based on animal testing do not always correlate with clinical risks. As a result, disconnection between in vitro studies, translational animal models, and human clinical studies decreases the effectiveness of the resulting therapeutic strategies. Further, animal testing is a slow and expensive process and raises ethical concerns. Thus, there is a significant need for lower cost, easy-to-use, robust, high-throughput biological models that can be used to predict human safety and efficacy of a candidate drug or pharmaceutical substance.

### SUMMARY OF THE INVENTION

In one aspect of the present invention, there is provided a method of deriving and maintaining endothelial cells, the method comprising: (a) culturing lateral plate mesoderm cells under oxygen-deprived condition to obtain endothelial lineage cells; and (b) culturing the endothelial lineage cells from (a) on an extracellular matrix to maintain and expand the endothelial lineage cells.

In another aspect, there is provided a cell co-culture system comprising an endothelial cell culture and a hepatocyte cell culture.

In a further aspect, there is provided a microfluidic-based system comprising the cell co-culture system of the present invention.

In yet a further aspect, there is provide a method of disease modelling or drug testing using the cell co-culture system or the microfluidic-based system of the present invention.

### BRIEF DESCRIPTION OF THE DRAWINGS

The invention will be better understood with reference to the detailed description when considered in conjunction with the non-limiting examples and the accompanying drawings, in which:

FIG. 1 shows the results of the generation and characterization of functional endothelial cells from human embryonic stem cells. FIG. 1A illustrates the timeline of endothelial induction from human pluripotent stem cells (hPSC), delineating the differentiation media and factors used. Human pluripotent stem cell-derived endothelial cells (HPSC-ECs) were derived via a lateral plate mesoderm population on day 5. By day 10, platelet and endothelial cell adhesion molecule 1 (PECAM1)-expressing cells could be isolated and expanded further. Abbreviations: BMP4, bone morphogenetic protein 4; EC, endothelial cell; FGF2, fibroblast growth factor 2; VEGF, vascular endothelial growth factor; LY294002, phosphoinositide 3-kinase inhibitor. FIG. 1B shows results of flow-cytometric analysis. The y-axis represents % of cells expressing PECAM1, while the x-axis represents the days of differentiation. The results demonstrate that 1% O₂ promoted higher percentages of PECAM1-expressing cells than 21% O₂, during endothelial induction from day 5 onward. ***, p<0.001 (compared with 21% O₂; n=3 independent biological replicates). FIG. 1C shows flow cytometric contour plots on the purity of PECAM1-expressing cells before and after FACS (fluorescence-activated cell sorting). The results demonstrate that the protocol used generated a sufficient yield of PECAM1+ cells for cell sorting on day 10 of differentiation, giving rise to a purity of 98.43±0.16%. FIG. 1D shows Western blot staining for endothelial adherens junction protein, CDH5, in cell lysates of hPSC-ECs and human coronary artery endothelial cells, HCAEC (human coronary artery endothelial cell, positive control). The results demonstrate the presence of CDH5 in hPSC-ECs. FIG. 1E shows immunostaining results for mature endothelial marker von Willebrand factor (vWF) in in hPSC-ECs, HCAEC (positive control) and HeLa cells (negative controls). The results demonstrate that the hPSC-ECs derived in the present application formed spontaneous tube structures that stained for the mature endothelial marker. FIG. 1F shows immunostaining for functional marker endothelial nitric oxide synthase (eNOS) in hPSC-ECs, HCAEC (positive control) and human hepatocellular carcinoma cells (HUH7 cells, negative control). FIG. 1G shows uptake of 3,3′-dioctadecyloxacarbocyanine-labeled acetylated low density lipoprotein (acetylated-LDL) in hPSC-ECs, HCAECs (positive control), and HeLa cells (negative control). Scale bars=100 μm. The results show that the hPSC-ECs derived in the present application were capable of taking up acetylated-LDL. FIG. 1H(i) shows phase contrast images of tube formation at 3 hour post-seeding. Scale bars=500 μm. FIG. 1H(ii) shows quantification of tube forming capability of cells. ***, p-value ≤0.001 relative to HeLa; ##, p-value ≤0.01 relative to HCAEC (n=3 independent biological replicates). The results demonstrate that there is comparable tube forming capability between the hPSC-ECs generated and HCAEC (positive control).

FIG. 2 shows inflammatory activation of H9-embryonic stem cell derived endothelial cells by IL-1β. FIG. 2A represents bar charts showing up regulation of inflammatory genes in human pluripotent stem cell-derived endothelial cells (hPSC-ECs) stimulated by IL-13 (20 ng/ml) for 6 hours. FIG. 2B represents immunostaining of NFκB showing nuclear translocation in hPSC-ECs after IL-13 (20 ng/ml) for 1 hour. Scale bars=100 μm. FIG. 2C represents bar charts showing enzyme-linked immunosorbent assay quantification of interleukin 8 (IL-8) concentrations in conditioned media of IL-1β-stimulated hPSC-ECs. *, p-value ≤0.05; **, p-value ≤0.01; ***, p-value ≤0.001 (compared with their respective unstimulated controls; n=3 independent biological replicates). The results demonstrate that the production of IL-8 from conditioned media of IL-1β-stimulated hPSC-ECs was significantly higher than that of the unstimulated cells. Abbreviations: IL, interleukin; NFκB 1, nuclear factor kB1.

FIG. 3 shows quercetin and genistein are unable to suppress IL-1β-induced inflammation in H9-embryonic stem cell derived endothelial cells (ECs). FIG. 3A shows gene expression of inflammatory markers in IL-1β-stimulated hPSC-ECs treated with quercetin and genistein over time. The results demonstrate that gene expression of inflammatory markers did not show substantial reduction from the IL-10 stimulated levels at various time points. FIG. 3B(i) shows immunostaining for NFκB in unstimulated and IL-1β-stimulated hPSC-ECs, treated with or without quercetin and genistein. Scale bars=100 μm. FIG. 3B(ii) shows Quantification of nuclear co-localization levels of NFκB by Pearson's coefficient. *** p-value ≤0.001 (compared with their respective unstimulated controls; n=3 independent biological replicates). The results demonstrate that NFκB nuclear translocation levels remained elevated despite administration of quercetin and genistein. FIG. 3C shows enzyme-linked immunosorbent assay quantification of IL-8 concentrations in conditioned media of IL-1b-stimulated hPSC-ECs, treated with or without quercetin and genistein for 48 hours. The results demonstrate that there was also no significant reduction of IL-8 protein levels from the conditioned media of stimulated hPSC-ECS after nutraceutical treatment for 48 hours. FIG. 3D, 3E show Liquid chromatography-mass spectrometry analysis of metabolic profiles of parent nutraceuticals, quercetin and genistein by hPSC-ECs. Primary and secondary metabolites of nutraceuticals were indicated. *, p-value ≤0.05; **, p-value ≤0.01 (compared with their respective 6-hour time points; n=3 independent biological replicates). Abbreviations: IL, interleukin; IS, internal standard; NFκB, nuclear factor kB.

FIG. 4 shows H9-embryonic stem cell-derived hepatocytes (HEPs) are able to effectively metabolize nutraceuticals. FIG. 4A represents immunocytochemistry showing presence of albumin in hPSC-HEPs and HUH7 cells (positive control), but not in HeLa cells (negative control). Scale bars=200 μm. FIG. 4B represents characterization of cytochrome P450 gene expression in hPSC-HEPs, PHH (primary human hepatocytes, positive control), and hPSC (negative control). FIGS. 4C and 4D show liquid chromatography-mass spectrometry analysis of metabolic profiles of parent nutraceuticals, quercetin and genistein by hPSC-HEPs. The results demonstrate that the production of primary and secondary metabolites of nutraceuticals increased with time, while the levels of both quercetin and genistein gradually declined over time in the presence of hPSC-HEPs. *, p-value ≤0.05; **, p-value ≤0.01 (compared with their respective 6-hour time points; n=3 independent biological replicates). FIG. 4E shows gene expression levels of inflammatory markers in IL-1β-stimulated hPSC-HEPs upon nutraceutical treatment for 48 hours. Quercetin or genistein treatments decreased most of the inflammatory marker expression in stimulated hPSC-HEPs. *, p-value ≤0.05; **, p-value ≤0.01; ***, p-value ≤0.001 (compared with stimulated group without nutraceutical treatment (black bar); n=3 independent biological replicates). FIG. 4F shows enzyme-linked immunosorbent assay quantification of IL-8 concentrations in conditioned media of IL-1β-stimulated hPSC-HEPs treated with or without quercetin and genistein for 48 hours. The results demonstrate that the production of IL-8 protein from conditioned media of hPSC-HEPs was significantly suppressed after treating with nutraceuticals for 48 hours. **, p-value ≤0.01 (compared with stimulated group without nutraceutical treatment; n=3 independent biological replicates). Abbreviations: CYP3A4, cytochrome P450 3A4.

FIG. 5 shows co-culture with H9-embryonic stem cell-derived hepatocytes (H9-ESC-HEPs) abrogates inflammatory activation in IL-1β-stimulated H9-embryonic stem cell-derived endothelial cells (H9-ESC-ECs). FIG. 5A represents schematics showing two experimental setups to study endothelial-hepatic paracrine interactions. FIG. 5B(i) shows immunostaining for NFκB in unstimulated and IL-1β-stimulated hPSC-ECs, treated with or without quercetin and genistein. Scale bars=100 μm. Fig. B(ii) shows bar charts demonstrating quantification of NFκB nuclear translocation. The results show that quercetin or genistein significantly decreased levels of NFκB nuclear colocalization in co-culture of hPSC-ECs with hPSC-HEPs. FIG. 5C represents bar charts showing IL-8 protein levels. It shows that IL-8 protein levels were significantly reduced in endothelial-hepatic co-culture but not in conditioned media setup. Statistical differences were compared with their respective stimulated groups without nutraceutical treatment. ***, p-value ≤0.001 (n=3 independent biological replicates).

FIG. 6 shows efficiency of endothelial differentiation in H9-ESCs. FIG. 6A shows expression of endothelial genes over time under 1% or 21% O₂ culture. The results show that endothelial genes were significantly enhanced in 1% O₂ as compared to 21% O₂, peaking primarily around day 10. Data is represented as fold change over unstimulated control±SEM. *, p-value ≤0.05, ***, p-value ≤0.001. FIG. 6B shows representative histogram plots of PECAM1-expressing cells during endothelial differentiation (day 10, 15, 20), under 1% O₂ vs 21% O₂ condition. The results show that endothelial specification was optimal around day 10 in 1% O₂, with more than 45% of the cells positive for PECAM1.

FIG. 7 shows results of functional characterization of endothelial cells from BJ and IMR90 induced pluripotent stem cell lines. FIG. 7A shows Western blot of CDH5 protein in HCAEC, BJ-EC, IMR90-EC (duplicates). Glycosylated forms of CDH5 were detected in BJ-EC and IMR90-EC. FIG. 7B shows immunostaining of vWF in BJ-EC, IMR90-EC and HeLa cells (negative control). Scale bars, 100 μm. FIG. 7C shows tube formation assay of BJ-EC and IMR90-EC seeded on matrigel plugs. The results show that the BJ- and IMR90-derived ECs also demonstrated tube formation capability. Scale bars, 500 μm.

FIG. 8 demonstrates the inflammatory activation in BJ-ECs and IMR90-ECs. FIGS. 8A and 8A(I) represents bar charts showing increased expression in inflammatory marker genes upon IL-13 stimulation in BJ-ECs and IMR90-ECs. Data is represented as fold change over unstimulated control±SD. **, p-value ≤0.01; ***, p-value ≤0.001. FIG. 8B(I) shows immunofluorescent images of NFκB nuclear translocation upon IL-1β stimulation in BJ-ECs (top four panels) and IMR90-ECs (bottom four panels). Scale bar=50 am. FIG. 8B(II) represents bar charts of quantification of immunofluorescent images showing significant increase in NFκB nuclear co-localization. *, p-value ≤0.05, ***, p-value ≤0.001. FIG. 8C represents bar charts showing increased IL-8 protein levels in the IL-1β-stimulated BJ-ECs and IMR90-ECs. ***, p-value ≤0.001.

FIG. 9 shows turnover of metabolites from quercetin (FIG. 9A, 9B) and genistein (FIG. 9C, 9D) in H9-ESC-HEPs and primary rat hepatocytes. Data is represented as fold change in the metabolite levels over parent metabolite at 6 hrs, fold change in peak ratio (metabolite peak/IS peak)±SD, n=3. The results demonstrate that quercetin and genistein declined over time in both hPSC-HEPs and freshly isolated primary rat hepatocytes, giving rise to metabolites.

FIG. 10 shows LC-MS analysis of quercetin and genistein metabolites in conditioned media and co-cultures. FIG. 10A shows metabolite profile of quercetin and genistein in hPSC-HEP conditioned media treated on hPSC-ECs. FIG. 10B shows metabolite profile of quercetin and genistein in co-cultured media of hPSC-ECs and hPSC-HEPs. The metabolite profiles of quercetin and genistein in each of the two configurations showed that there were detectable levels of various metabolites in the endothelial-hepatic co-culture but not in the conditioned media setting.

FIG. 11 shows functional characterization of H9-ESC-HEPs co-cultured with H9-ESC-ECs. FIG. 11A represents albumin immunofluorescence in hPSC-HEPs in monoculture and co-culture, showing similar levels of expression in both the culture configurations. Scale bar=100 m. FIG. 11B represents bar charts showing that the hPSC-HEPs co-cultured with hPSC-ECs express higher levels of CYP genes than the hPSC-HEPs in monoculture, supporting the beneficial effects of endothelial cells on hPSC-HEPs metabolic potential. n=3. Data is represented as fold change over undifferentiated hPSCs±SD. **, p-value ≤0.01, ***, p-value ≤0.001.

### DETAILED DESCRIPTION OF THE PRESENT INVENTION

The inventors of the present disclosure have set out to provide alternative biological model which could be used to predict human safety and efficacy of a candidate drug or pharmaceutical substance, in particular the safety and efficacy on vascular cells.

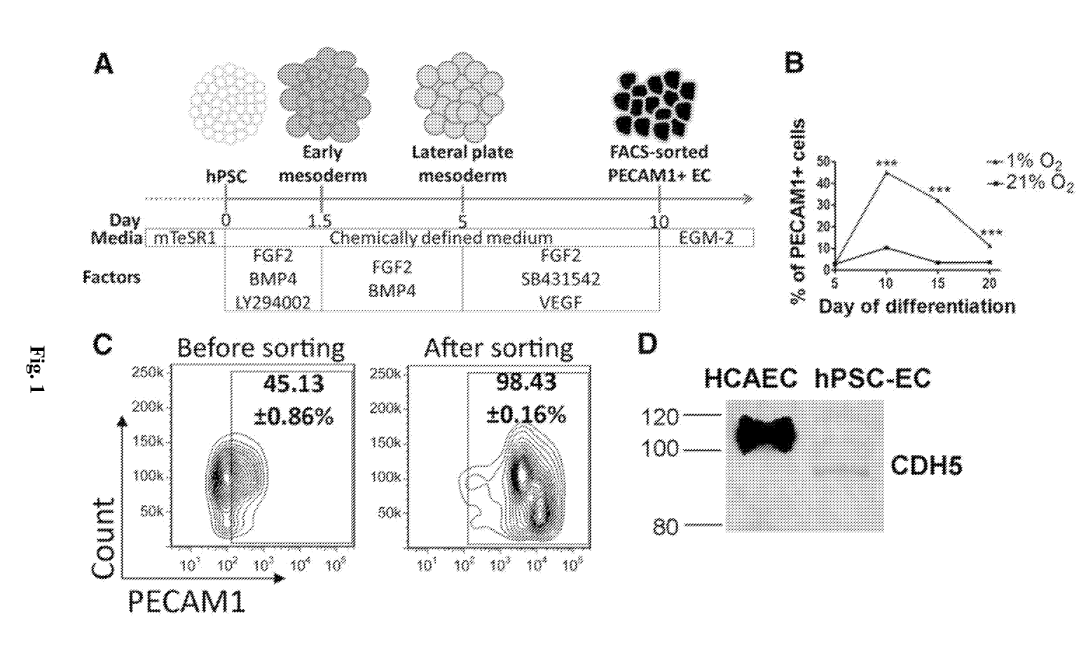

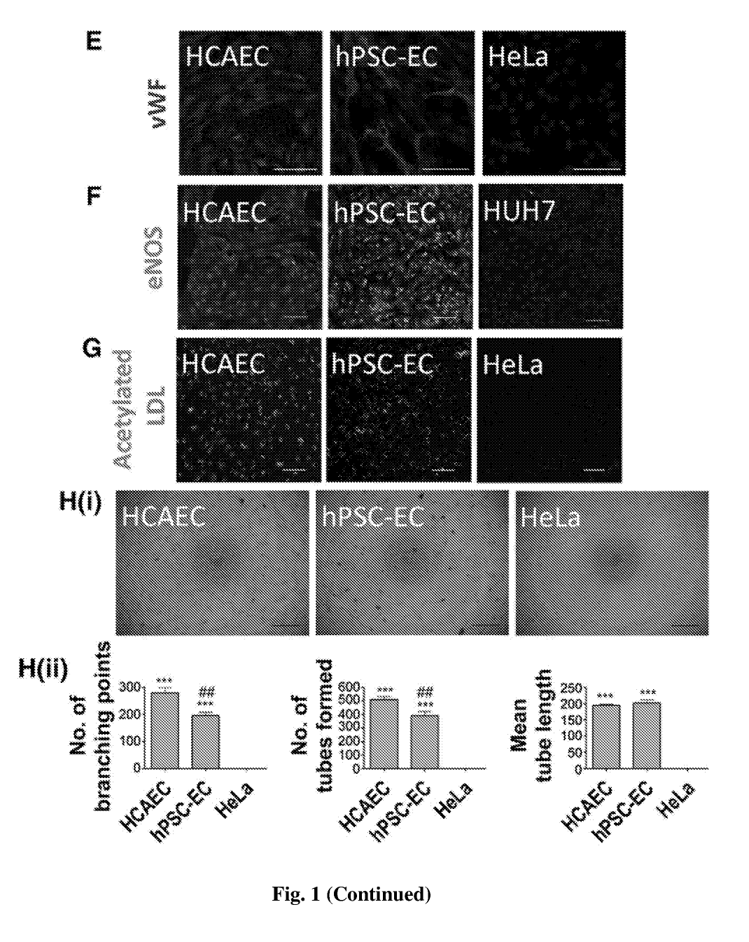

In a first aspect, there is provided a method of deriving and maintaining endothelial cells comprising: (a) culturing lateral plate mesoderm cells under oxygen-deprived condition to obtain endothelial lineage cells; and culturing the endothelial lineage cells from (a) on an extracellular matrix to maintain and expand the endothelial lineage cells. For example, referring to FIG. 1A, this method is directed to the culturing of the lateral plate mesoderm cells from day 5 onwards.

The term “endothelial cells” as used herein refers to the thin layer of squamous cells that form the endothelium. The term “endothelium” as used herein refers to a type of epithelium that lines the interior surface of blood vessels and lymphatic vessels, forming an interface between circulating blood or lymph in the lumen and the rest of the vessel wall. Endothelial cells in direct contact with blood are called vascular endothelial cells, whereas those in direct contact with lymph are known as lymphatic endothelial cells. Vascular endothelial cells line the entire circulatory system, from the heart to the smallest capillaries. These cells have unique functions in vascular biology. These functions include fluid filtration, such as in the glomerulus of the kidney, blood vessel tone, hemostasis, neutrophil recruitment, and hormone trafficking. Endothelium is mesodermal in origin. Endothelial cells are involved in many aspects of vascular biology, including barrier function, blood clotting (thrombosis & fibrinolysis), inflammation, formation of new blood vessels (angiogenesis), vasoconstriction and vasodilation (and hence the control of blood pressure). Endothelial dysfunction, or the loss of proper endothelial function, is a hallmark for vascular diseases, and is often regarded as a key early event in the development of atherosclerosis. Impaired endothelial function, causing hypertension and thrombosis, is often seen in patients with coronary artery disease, diabetes mellitus, hypertension, hypercholesterolemia, as well as in smokers. Endothelial dysfunction has also been shown to be predictive of future adverse cardiovascular events, and is also present in inflammatory disease such as rheumatoid arthritis and systemic lupus erythematosus.

The term “endothelial lineage cells” as used herein refers to cells that are committed to be developed into endothelial cells. Endothelial lineage cells or endothelial cells could be characterized by specific endothelial cell markers. Thus, in some examples, the endothelial lineage cells obtained from (a) and to be cultured on an extracellular matrix in (b) are characterized by and/or sorted based on the expression of one or more markers. These one or more markers include at least one of the following: PECAM-1, CD144 (VE-cadherin), Von Willebrand factor (vWF) and Endothelial NOS (eNOS), and can also include other endothelial cell markers. In some examples, the endothelial lineage cells are characterized by one, or two, or three, or all four of the above-mentioned markers. When more than one marker is used to characterize the endothelial lineage cells, the characterization could include the use of any of the following non-limiting examples of combination of markers: combination of PECAM-1, VE-cadherin, vWF and eNOS; combination of PECAM-1 and VE-cadherin; combination of PECAM-1 and vWF; combination of PECAM-1 and eNOS; combination of VE-cadherin and vWF; combination of VE-cadherin and eNOS; combination of vWF and eNOS; combination of PECAM-1, VE-cadherin and vWF; combination of PECAM-1, VE-cadherin and eNOS; combination of PECAM-1, vWF and eNOS; combination of VE-cadherin, vWF and eNOS. In some examples, the endothelial lineage cells are only characterized by one or more of the above-mentioned markers without the need of using other endothelial cell markers. In one specific example, the endothelial lineage cells only need to be characterized by PECAM-1. Additional characterization of endothelial lineage cells could be carried out by measuring the uptake of fluorescently-labelled acetylated low-density lipoprotein.

The term “PECAM-1” as used herein refers to platelet endothelial cell adhesion molecule, is also known as cluster of differentiation 31 (CD31). It is a protein that in human is encoded by the PECAM1 gene found on chromosome 17. PECAM-1 is found on the surface of platelets, monocytes, neutrophils, and some types of T-cells, and makes up a large portion of endothelial cell intercellular junctions. The encoded protein is a member of the immunoglobulin superfamily and is likely involved in leukocyte transmigration, angiogenesis, and integrin activation. In immunohistochemistry, it is used primarily to demonstrate the presence of endothelial cells in histological tissue sections.

The term “VE-cadherin” or “vascular endothelial cadherin” or “CD144” as used interchangeably herein refers to the cadherin encoded by the human gene CDH5. It is a classical cadherin from the cadherin superfamily and the CDH5 gene is located in a six-cadherin cluster in a region on the long arm of chromosome 16 that is involved in loss of heterozygosity events in breast and prostate cancer. The encoded protein is a calcium-dependent cell-cell adhesion glycoprotein composed of five extracellular cadherin repeats, a transmembrane region and a highly conserved cytoplasmic tail. Functioning as a classic cadherin by imparting to cells the ability to adhere in a homophilic manner, the protein may play an important role in endothelial cell biology through control of the cohesion and organization of the intercellular junctions.

The term “Von Willebrand factor” or “vWF” as used herein refers to a large multimeric glycoprotein present in blood plasma and produced constitutively as ultra-large vWF in endothelium (in the Weibel-Palade bodies), megakaryocytes (α-granules of platelets), and sub-endothelial connective tissue. The basic vWF monomer is a 2050-amino acid protein. Monomers are subsequently N-glycosylated, arranged into dimers in the endoplasmic reticulum and into multimers in the Golgi apparatus by crosslinking of cysteine residues via disulfide bonds. Multimers of vWF can be extremely large, >20,000 kDa, and consist of over 80 subunits of 250 kDa each. Only the large multimers are functional. vWF binds to a number of cells and molecules: (i) Factor VIII is bound to vWF while inactive in circulation, and it degrades rapidly when not bound to vWF; (ii) vWF binds to collagen, for example, when it is exposed in endothelial cells due to damage occurring to the blood vessel. Endothelium also releases vWF which forms additional links between the platelets' glycoprotein Ib/IX/V and the collagen fibrils; (iii) vWF binds to platelet gpIb when it forms a complex with gpIX and gpV. This binding occurs under all circumstances, but is most efficient under high shear stress (i.e., rapid blood flow in narrow blood vessels); (iv) vWF binds to other platelet receptors when they are activated, for example, by thrombin (i.e., when coagulation has been stimulated). vWF plays a major role in blood coagulation. Therefore, vWF deficiency or dysfunction (von Willebrand disease) leads to a bleeding tendency, which is most apparent in tissues having high blood flow shear in narrow vessels.

The term “Endothelial NOS” or “eNOS” as used interchangeably herein is also known as nitric oxide synthase 3 (NOS3) or constitutive NOS (cNOS). It is an enzyme that in humans is encoded by the NOS3 gene located in the 7q35-7q36 region of chromosome 7. This enzyme is one of three isoforms that synthesize nitric oxide (NO), a small gaseous and lipophilic molecule that participates in several biological processes. The other isoforms include neuronal nitric oxide synthase (nNOS), which is constitutively expressed in specific neurons of the brain and inducible nitric oxide synthase (iNOS), whose expression is typically induced in inflammatory diseases. eNOS is primarily responsible for the generation of NO in the vascular endothelium at the interface between circulating blood in the lumen and the remainder of the vessel wall. NO produced by eNOS in the vascular endothelium plays crucial roles in regulating vascular tone, cellular proliferation, leukocyte adhesion, and platelet aggregation. eNOS is a dimer containing two identical monomers of 134 kD constituted by a reductase domain, which displays binding sites for nicotinamide adenine dinucleotide phosphate (NADPH), flavin mononucleotide (FMN), and flavin adenine dinucleotide (FAD), and an oxidase domain, which displays binding sites for heme group, zinc, the cofactor tetrahydrobiopterin (BH4), and the substrate L-arginine. The reductase domain is linked to the oxidase domain by a calmodulin-binding sequence. In the vascular endothelium, NO is synthesized by eNOS from L-arginine and molecular oxygen, which binds to the heme group of eNOS, is reduced and finally incorporated into L-arginine to form NO and L-citrulline. The binding of the cofactor BH4 is essential for eNOS to efficiently generate NO. In the absence of this cofactor, eNOS shifts from a dimeric to a monomeric form, thus becoming uncoupled. In this conformation, instead of synthesizing NO, eNOS produces superoxide anion, a highly reactive free radical with deleterious consequences to the cardiovascular system. eNOS has a protective function in the cardiovascular system, which is attributed to NO production. Regulation of the vascular tone is one of the best known roles of NO in the cardiovascular system. Once produced in endothelial cells, NO diffuses across the vascular smooth muscle cell membranes and activates the enzyme soluble guanylate cyclase (sGC), which catalyzes the conversion of guanosine triphosphate into cyclic guanosine monophosphate (cGMP). cGMP, in turn, activates protein kinase G (PKG), which promotes multiple phosphorylation of cellular targets lowering cellular Ca²⁺ concentrations and promoting vascular relaxation. NO exerts anti-proliferative effects by cGMP-dependent inhibiting Ca²⁺ influx or by directly inhibiting the activity of arginase and ornithine decarboxylase, decreasing the generation of polyamides required for DNA synthesis. NO also has antithrombotic effects that result of its diffusion across platelet membrane and sGC activation, resulting in inhibition of platelet aggregation. Moreover, NO affects leukocyte adhesion to the vascular endothelium by inhibiting the nuclear factor kappa B (NFκB), which induces vascular endothelial expression of chemokines and adhesion molecules. In addition to these functions, NO produced by eNOS has antioxidant properties as it reduces superoxide anion formation as a result of NO-induced increases in the expression of superoxide dismutase, an antioxidant enzyme that catalyzes the conversion of superoxide anion to hydrogen peroxide. Furthermore, part of antioxidants properties of NO is attributable to up-regulation of heme-oxygenase-I and ferritin expression, which reduce superoxide anion concentrations in blood vessels.

The term “stem cell” as used herein refers to undifferentiated biological cells that are capable of differentiating into more specialized cells and that are capable of dividing (through mitosis) to produce more stem cells. Stem cells are found in multicellular organisms. In mammals, there are two broad types of stem cells: embryonic stem cells, which are isolated, for example, from the inner cell mass of blastocysts, and adult stem cells, which are found in various tissues. In adult organisms, stem cells and progenitor cells function as a repair system for the body by replenishing adult tissues. In a developing embryo, stem cells can differentiate into all the specialized cells derived from any one of the three primary germ layers, namely ectoderm, endoderm and mesoderm, present in the early stages of embryonic development. Stem cells can also maintain the normal turnover of regenerative organs, such as blood, skin, or intestinal tissues in a developing embryo.

The three commonly known, accessible sources of autologous adult stem cells in humans are the bone marrow, which requires extraction by harvesting cells, usually from the femur or iliac crest; adipose tissue (lipid cells), which requires extraction by liposuction, and blood, which requires extraction, usually through a apheresis machine. Stem cells can also be taken from umbilical cord blood just after birth. Of all stem cell types, autologous harvesting involves the least risk. By definition, autologous cells are obtained from one's own body.

Any mammalian stem cell can be used in accordance with the methods of the invention as disclosed herein, including but not limited to, stem cells isolated from cord blood, placenta and other sources. The stem cells may be isolated from any mammalian species, for example, but not limited to, mouse, rat, rabbit, guinea pig, dog, cat, pig, sheep, cow, horse, monkey and human. In one example, the stem cells are obtained from a human. The stem cells may include pluripotent cells, which are cells that have complete differentiation versatility, that are self-renewing, and can remain dormant or quiescent within tissue. The stem cells may also include multipotent cells or committed progenitor cells. In one example, the method as disclosed herein is performed without the use of human embryonic stem cells. Instead of human embryonic stem cells, other types of pluripotent cells can be used in accordance with the present invention. In another example, the method as disclosed herein is performed on induced pluripotent stem cells. In yet another example, the method as disclosed herein is performed using embryonic stem cells that are not of human origin.

As used herein, the term “pluripotent stem cell” or “pluripotent cell” refers to a stem cell that has the potential to differentiate into any of the three germ layers: the endoderm, from which, for example, the interior stomach lining, gastrointestinal tract and the lungs develop; the mesoderm, from which, for example, muscle, bone, blood and urogenital structures develop; or the ectoderm, from which, for example, epidermal tissues and nervous system develop. It is noted however, that cell pluripotency is considered to be a continuum, ranging from the completely pluripotent cell that can form every cell of the embryo proper, for example, embryonic stem cells and induced pluripotent stem cells, to the incompletely or partially pluripotent cell that can form cells of all three germ layers but that may not exhibit all the characteristics of completely pluripotent cells.

Pluripotent stem cells undergo further specialization into multipotent cells that then give rise to functional cells. The term “multipotent cell” as used herein refers to unspecialized cells that have the ability to self-renew for long periods of time and differentiate into specialized cells with specific functions. A multipotent cell can give rise to other types of cells but it is limited in its ability to differentiate, as compared to pluripotent cells. Hence, adult stem cells are generally considered as multipotent cells because their specialization potential is limited to one or more cell lines. A multipotent cell can be a multipotent stem cell or a multipotent progenitor cell. The term “progenitor cell” as used herein refers to a biological cell that has a tendency to differentiate into a specific type of cell. The most important difference between stem cells and progenitor cells is that stem cells can replicate indefinitely, whereas progenitor cells can divide only a limited number of times. Examples of multipotent progenitor cells include, but are not limited to, radial glial cells (embryonic neural stem cells) that give rise to excitatory neurons in the fetal brain through the process of neurogenesis; hematopoietic stem cells (adult stem cells) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; and muscle satellite cells (progenitor cells) that contribute to differentiated muscle tissue.

Each specialized cell type in an organism expresses a subset of all the genes that constitute the genome of that species. Each cell type is defined by its particular pattern of regulated gene expression. Cell differentiation is thus a transition of a cell from one cell type to another and, as a result, involves a switch from one pattern of gene expression to another. Cellular differentiation during development can be understood as the result of a gene regulatory network.

The term “mesoderm” as used herein refers to one of the three primary germ layers in the very early embryo. The other two layers are the ectoderm (outside layer) and endoderm (inside layer), with the mesoderm as the middle layer between them. It is formed through a process called gastrulation. There are three important components, the paraxial mesoderm, the intermediate mesoderm and the lateral plate mesoderm. The paraxial mesoderm forms the somitomeres, which give rise to mesenchyme of the head and organize into somites in occipital and caudal segments, forming sclerotome (cartilage and bone), and dermatome (subcutaneous tissue of the skin). Signals for somite differentiation are derived from surroundings structures, including the notochord, neural tube and epidermis. The intermediate mesoderm connects the paraxial mesoderm with the lateral plate, eventually it differentiates into urogenital structures consisting of the kidneys, gonads, their associated ducts, and the adrenal glands.

The term “lateral plate mesoderm” as used herein refers to a type of mesoderm that is found at the periphery of the embryo, it is a precursor tissue of vascular lineages that could give rise to the heart, blood vessels and blood cells of the circulatory system as well as to the mesodermal component of the limbs. The lateral plate mesoderm is split into two layers, the somatic lateral plate mesoderm, which forms the future body wall, and the splanchnic lateral plate mesoderm, which forms the circulatory system. In one example, the lateral plate mesoderm used in the method of the present disclosure comprises splanchnic lateral plate mesoderm cells. In some examples, the lateral plate mesoderm cells are obtained by (i) culturing pluripotent stem cells to obtain early mesodermal precursor cells; and (ii) culturing the early mesodermal precursor cells from (i) to obtain lateral plate mesoderm cells. In some examples, the pluripotent stem cells used in (i) to obtain early mesodermal precursor cells can be embryonic stem cells or induced pluripotent stem cells. In some examples, the early mesoderm differentiation step (i) can be carried out using a chemically defined medium, supplemented with fibroblast growth factor, TGF-β inhibitor and phosphoinositide 3-kinase (PI3K) inhibitor. In one specific example, the fibroblast growth factor used is FGF2, the TGF-β inhibitor used is bone morphogenetic protein 4 (BMP4) and the PI3K inhibitor used is LY294002, as exemplified by the method described in Example 1 of the present application.

The term “oxygen-deprived condition” or “hypoxia” as used interchangeably herein refers to the condition under which the cells are cultured under an inadequate supply of oxygen. In typical cell cultures, atmospheric oxygen conditions (about 21% O₂) are used. Under oxygen-deprived condition or hypoxia, the oxygen supply can be between about 0 to 10%, or between about 0.5 to 9%, or between about 1 to 8%, or between about 1.5 to 7%, or between about 2 to 6%, or between about 2.5 to 5%, or between about 3 to 4% oxygen, or at about 0.5, 1, 1.5, 2, 2.5, 3, 3.5, 4, 4.5, 5, 5.5, 6, 6.5, 7, 7.5, 8, 8.5, 9, 9.5 or 10% oxygen. In one specific example, the oxygen-deprived condition is carried out at 1% oxygen. Oxygen-deprived condition can increase efficiency of endothelial differentiation, as up-regulation of hypoxia-inducible factor triggers downstream targets that play an important role in early blood vessel development.

Cell culture is the process by which cells are grown under controlled conditions that mimic their natural environment but are outside of their natural environment. Cell culture conditions vary for each cell type, but the required artificial environments usually consist of a suitable vessel with one or more substrates or one or more media that supply the essential nutrients (such as amino acids, carbohydrates, vitamins and minerals), growth factors, hormones, and gases (usually CO₂ and/or O₂) required for cell growth, and that regulate the physio-chemical environment (for example, pH buffer, osmotic pressure, temperature, humidity and the like). Most cells require a surface or an artificial substrate on which to grow on (also known as adherent or monolayer culture), whereas others can be grown free-floating in culture medium (also known as suspension culture), usually under agitation (for example, roller bottle culture and the like). In practice, the term “cell culture” refers to the culturing of cells derived from multicellular eukaryotes, especially animal cells as well as diseased human tissue, in contrast with other types of culture that also grow cells, such as plant tissue culture, fungal culture, microbiological culture (of microbes), and viral culture with the cells being used as hosts for, for example, viral replication.

The method of the first aspect allows the lateral plate mesoderm cells to differentiate into endothelial cells. Cellular differentiation is the process describing the change of a cell from one cell type to another. Most commonly, the cell changes to a more specialized type. Differentiation occurs numerous times during the development of a multicellular organism, as it changes from a simple zygote to a complex system of tissues and cell types. Differentiation continues in adulthood, as adult stem cells divide and create fully differentiated daughter cells during tissue repair and during normal cell turnover. Differentiation dramatically changes a cell's size, shape, membrane potential, metabolic activity, and responsiveness to signals. These changes are largely due to highly controlled modifications in gene expression and are the study of the field of epigenetics. With a few exceptions, cellular differentiation almost never involves a change in the DNA sequence itself. Thus, different cells can have very different physical characteristics, usually separated temporally, despite having the same genome.

After the stem cells differentiate into the desired type of cells, such as endothelial cells, the differentiated cells have to be maintained. There are a variety of platforms that can be used to maintain the differentiated cells, which include but are not limited to, scaffold systems such as hydrogel matrices and solid scaffolds, and scaffold-free systems such as low-adhesion plates, nanoparticle facilitated magnetic levitation, and hanging drop plates. In one example, the method disclosed herein uses an extracellular matrix to maintain and expand the endothelial cells obtained.

As used herein, the term “ECM” or “extracellular matrix” refers to a collection of extracellular molecules secreted by cells that provides structural and biochemical support to the surrounding cells. Because multicellularity evolved independently in different multicellular lineages, the composition of ECM varies between multicellular structures. However, cell adhesion, cell-to-cell communication and differentiation are common functions of the ECM.

Animal extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the extracellular matrix (ECM). Basement membranes are sheet-like depositions of extracellular matrix, on which various epithelial cells rest. Each type of connective tissue in animals has a type of extracellular matrix: collagen fibres and bone mineral comprise the extracellular matrix of bone tissue; reticular fibres and ground substance comprise the extracellular matrix of loose connective tissue; and blood plasma is the extracellular matrix of blood.

The plant extracellular matrix includes cell wall components, like cellulose, in addition to more complex signalling molecules. Some single-celled organisms adopt multicellular biofilms in which the cells are embedded in an extracellular matrix composed primarily of extracellular polymeric substances (EPS). Due to its diverse nature and composition, the extracellular matrix can serve many functions, such as providing support, segregating tissues from one another, and regulating intercellular communication. The extracellular matrix regulates a cell's dynamic behaviour. In addition, it sequesters a wide range of cellular growth factors and acts as local store for them. Changes in physiological conditions can trigger protease activities that cause local release of such stores. This allows the rapid and local growth factor-mediated activation of cellular functions without the requirement of de novo synthesis.

The stiffness and elasticity of the extracellular matrix has important implications in cell migration, gene expression, and differentiation. Cells actively sense extracellular matrix rigidity and migrate preferentially towards stiffer surfaces in a phenomenon called durotaxis. Cells also detect elasticity and can adjust their gene expression. Components of the extracellular matrix are produced intracellularly by resident cells and secreted into the extracellular matrix via exocytosis. Once secreted, they then aggregate with the existing matrix. The extracellular matrix is composed of an interlocking mesh of fibrous proteins and glycosaminoglycans (GAGs). Thus, an extracellular matrix can comprise, but is not limited to, proteoglycans (for example, heparan sulfate, chrondroitin sulfate, keratin sulfate), nonproteoglycan polysaccharides (for example, hyaluronic acid), proteins (for example, collagen, elastin) and other components, such as, but not limited to fibronectin and laminin.

Therefore, in one example, the extracellular matrix promotes cell differentiation and/or can maintain three-dimensional culture and/or promotes the development of complex tissue. Thus, the material(s) from which comprise the extracellular matrix is/are but is/are not limited to, matrigel, gelatine, methylcellulose, collagen, alginate, alginate beads, agarose, fibrin, fibrin glue, fibrinogen, blood plasma fibrin beads, whole plasma or components thereof, laminins, fibronectins, protecogylcans, HSP, chitosan, heparin, other synthetic polymer or polymer scaffolds and solid support materials. In one example, the extracellular matrix is made of one or more of the following: collagen, gelatin, fibronectin and laminins.

A cell culture media used in cell culture usually comprises at least the following: a carbon or carbohydrate source (for example glucose or glutamine) as a source of energy; amino acids for protein synthesis; vitamins, which promote cell survival and growth; a balanced salt solution, usually a mixture of various ions to maintain optimal osmotic pressure within the cells and to act as cofactors for various cofactor-mediated reactions (for example cell adhesion, enzymatic reactions and the like); a pH indicator (for example phenol red; indicating a change in pH from neutral to basic or acidic, which usually indicate the presence of nutrient depletion, contamination, accumulation of necrotic cells and the like) and a buffer (for example, bicarbonate or HEPES buffer) to maintain the required pH in the media. In addition to the components listed above, cell culture media can and usually is modified by the person skilled in the art to their desired requirements in cell culture. For example, the use of fetal calf or bovine serum is required for the growth and maintenance of some cell lines in vitro, but is not required for some and is to be avoided in others, for example when serum-starved cells are required for cytokine analysis. As defined in the art, a “defined medium” (also known as “chemically defined medium” or “synthetic medium”) is a cell culture medium in which all the chemicals used are known and no yeast, animal, or plant tissue is present.

In some examples, the method of the present invention involves the use of a first culture medium in which the lateral plate mesoderm cells are cultured in. In some examples, the lateral plate mesoderm cells are cultured in the first cell culture medium for 3 to 10 days, or 3 to 7 days, or 3 to 5 days, or 5 to 10 days, or 5 to 7 days, or 7 to 10 days, or for 1, 2, 3, 4, 5, 6, 7, 8, 9 or 10 days. In one specific example, the lateral plate mesoderm cells are cultured in the first cell culture medium for 5 days.

In one example, the first culture medium comprises the following amongst other components: fibroblast growth factor, TGF-β inhibitor and vascular endothelial growth factor (VEGF).

The term “fibroblast growth factor” or the short form “FGF” as used herein refers to a family of growth factors, with members involved in angiogenesis, wound healing, embryonic development and various endocrine signalling pathways. The fibroblast growth factors are heparin-binding proteins and interactions with cell-surface-associated heparan sulfate proteoglycans have been shown to be essential for fibroblast growth factor signal transduction. Fibroblast growth factors are key players in the processes of proliferation and differentiation of wide variety of cells and tissues. Fibroblast growth factors are multifunctional proteins with a wide variety of effects; they are most commonly mitogens but also have regulatory, morphological, and endocrine effects. They have been alternately referred to as “pluripotent” growth factors and as “promiscuous” growth factors due to their multiple actions on multiple cell types. Promiscuous refers to the biochemistry and pharmacology concept of how a variety of molecules can bind to and elicit a response from single receptor. In the case of fibroblast growth factor, four receptor subtypes can be activated by more than twenty different fibroblast growth factor ligands. Thus the functions of FGFs in developmental processes include mesoderm induction, antero-posterior patterning, limb development, neural induction and neural development, and in mature tissues/systems angiogenesis, keratinocyte organization, and wound healing processes.

Fibroblast growth factors are critical during normal development of both vertebrates and invertebrates and any irregularities in their function leads to a range of developmental defects. One important function of FGF1 and FGF2 is the promotion of endothelial cell proliferation and the physical organization of endothelial cells into tube-like structures. They thus promote angiogenesis, the growth of new blood vessels from the pre-existing vasculature. FGF1 can be used to induce angiogenesis in the heart, as well as stimulating blood vessel growth. FGF1 and FGF2 stimulate angiogenesis and the proliferation of fibroblasts that give rise to granulation tissue, which fills up a wound space/cavity early in the wound-healing process. FGF7 and FGF10 (also known as keratinocyte growth factors KGF and KGF2, respectively) stimulate the repair of injured skin and mucosal tissues by stimulating the proliferation, migration and differentiation of epithelial cells, and they have direct chemotactic effects on tissue re-modelling. Another fibroblast growth factor family member, FGF8, regulates the size and positioning of the functional areas of the cerebral cortex (Brodmann's Areas). FGFs are also important for maintenance of the adult brain.

Members of the FGF19 subfamily (FGF15, FGF19, FGF21, and FGF23) can act in an endocrine fashion on far-away tissues, such as intestine, liver, kidney, adipose, and bone. For example, FGF15 and FGF19 (FGF15/19) are produced by intestinal cells but act on FGFR4-expressing liver cells to down-regulate the key gene (CYP7A1) in the bile acid synthesis pathway. FGF23 is produced by bone but acts on FGFR1-expressing kidney cells to regulate the synthesis of vitamin D and phosphate homeostasis. In humans, 22 members of the FGF family have been identified, all of which are structurally related signalling molecules. FGF1 through to FGF10 are known to all bind fibroblast growth factor receptors (FGFRs). FGF1 is also known as acidic, and FGF2 is also known as basic fibroblast growth factor. FGF11, FGF12, FGF13, and FGF14, also known as FGF homologous factors 1 to 4 (FHF1-FHF4), have been shown to have distinct functions compared to the FGFs. Although these factors possess remarkably similar sequence homology, they do not bind FGFRs and are involved in intracellular processes unrelated to the FGFs This group is also known as “iFGF”. Human FGF18 is involved in cell development and morphogenesis in various tissues including cartilage. Human FGF20 was identified based on its homology to Xenopus FGF-20 (XFGF-20). FGF15 through FGF23 were described later and functions are still being characterized. FGF15 is the mouse ortholog of human FGF19 (there is no human FGF15) and, where their functions are shared, they are often described as FGF15/19. In contrast to the local activity of the other FGFs, FGF15/19, FGF21 and FGF23 have systemic effects. The crystal structures of FGF1 have been solved and found to be related to interleukin 1-beta. Both families have the same beta trefoil fold consisting of 12-stranded beta-sheet structure, with the beta-sheets are arranged in 3 similar lobes around a central axis, 6 strands forming an anti-parallel beta-barrel. In general, the beta-sheets are well-preserved and the crystal structures superimpose in these areas. The intervening loops are less well-conserved—the loop between beta-strands 6 and 7 is slightly longer in interleukin-1 beta.

In one example of the present disclosure, the fibroblast growth factor (FGF) used in the first culture medium binds to fibroblast growth factor receptors (FGFRs). The FGF can be, for example, FGF1, FGF2, FGF3, FGF4, FGF5, FGF6, FGF7, FGF8, FGF9 and FGF10. In one specific example, the FGF is FGF2.

In some examples, the fibroblast growth factor is at a concentration of between about 0.5 to about 300 ng/ml, or between about 1 to about 250 ng/ml, or between about 5 to about 200 ng/ml, or between about 10 to about 150 ng/ml, or between about 15 to about 100 ng/ml, or between about 20 to about 90 ng/ml, or between about 25 to about 80 ng/ml, or between about 30 to about 70 ng/ml, or between about 35 to about 60 ng/ml, or between about 40 to about 50 ng/ml, or at about 1, 2, 3, 4, 5, 6, 7, 8, 9, 10, 12.5, 15, 17.5, 20, 22.5, 25, 27.5, 30, 40, 50, 60, 70, 80, 90, 100, 125, 150, 175, 200, 225, 250, 275 or 300 ng/ml. In one specific example, the fibroblast growth factor is at a concentration of 4 ng/ml.

The term “inhibitor” as used herein refers to a molecule or compound that is capable of decreasing, down-regulating or, in some cases, completely ceasing, activity of a target molecule. An inhibitor is usually characterised and named for its target; for example, a compound that binds to an enzyme and thereby decreases its activity is called an enzyme inhibitor. An inhibitor can be either reversible or irreversible, meaning that in terms of binding to its target, this target binding may be subsequently broken (reversible) or not (irreversible). Inhibition of a target molecule can, for example, be competitive, uncompetitive, non-competitive, or mixed. Conversely, a molecule or compound that is capable of increasing, up-regulating or initiating the activity of a target molecule is known as an activator.

As uses herein, the term “TGF-β inhibitor” refers to a molecule or compound that is capable of blocking or down regulating the effect of TGF-β. TGF-β is a polypeptide member of the transforming growth factor beta superfamily of cytokines. It is a secreted protein that performs many cellular functions, including, but not limited to, the control of cell growth, cell proliferation, cell differentiation and apoptosis. In humans, TGF-β1 is encoded by the TGFB1 gene. Other members of this superfamily include, but are not limited to, bone morphogenetic proteins (BMPs), growth and differentiation factors (GDFs), anti-miillerian hormone (AMH), Activin (for example, Activin A, B and AB), Nodal and different TGF-β's (for example, TGFβ-1, TGFβ-2, TGFβ-3).

TGF-β and the related proteins of said transforming growth factor beta superfamily of cytokines are involved in the so-called TGF-β signalling pathway. This pathway is involved in many cellular processes in both the adult organism and the developing embryo, including, but not limited to, cell growth, cell differentiation, apoptosis, cellular homeostasis and other cellular functions. In spite of the wide range of cellular processes that the TGF-β signalling pathway regulates, the process is relatively straightforward. TGF-β superfamily ligands bind to a type II receptor (usually a serine/threonine receptor kinase), which recruits and phosphorylates a type I receptor. The type I receptor then phosphorylates receptor regulated SMADs (R-SMADs) which can now bind the co-SMAD SMAD4 (also known as SMAD family member n° 4, Mothers against decapentaplegic homolog 4, JIP, MADH4, MYHRS, or DPC4 (Deleted in Pancreatic Cancer-4)). R-SMAD/co-SMAD complexes accumulate in the nucleus where they act as transcription factors and participate in the regulation of target gene expression.

Thus, in one example, the TGF-β inhibitor is characterized by at least one or more of the following characteristics, which are, but are not limited to, inhibition of TGF-β type I receptor ALK5 kinase; inhibition of type I Activin/Nodal receptor ALK4; inhibition of type I Nodal receptor ALK7; inhibition of SMAD2/3 phosphorylation; and/or inhibition of the Activin/TGF β/SMAD signalling pathway.

Examples of TGF-β inhibitors include but are not limited to small molecule TGF-β inhibitors and antibodies of TGF-β receptor. Exemplary small molecule TGF-β inhibitors include but are not limited to: 4-[4-(1,3-benzodioxol-5-yl)-5-(2-pyridinyl)-1H-imidazol-2-yl]benzamide (SB 431542), 3-(6-Methyl-2-pyridinyl)-N-phenyl-4-(4-quinolinyl)-1H-pyrazole-1-carbothioamide (A 83-01), 2-(3-(6-Methylpyridine-2-yl)-1H-pyrazol-4-yl)-1,5-naphthyridine (RepSox), 6-[2-(1,1-Dimethylethyl)-5-(6-methyl-2-pyridinyl)-1H-imidazol-4-yl]quinoxaline (SB525334), 2-(5-Chloro-2-fluorophenyl)-4-[(4-pyridyl)amino]pteridine (SD208), 2-[4-(1,3-Benzodioxol-5-yl)-2-(1,1-dimethylethyl)-1H-imidazol-5-yl]-6-methyl-pyridine (SB505124), 4-[4-(2,3-Dihydro-1,4-benzodioxin-6-yl)-5-(2-pyridinyl)-1H-imidazol-2-yl]benzamide (D 4476), 4-[3-(2-Pyridinyl)-1H-pyrazol-4-yl]-quinoline (LY364947), 4-[2-Fluoro-5-[3-(6-methyl-2-pyridinyl)-1H-pyrazol-4-yl]phenyl]-1H-pyrazole-1-ethanol (R268712) and 4-[4-[3-(2-Pyridinyl)-1H-pyrazol-4-yl]-2-pyridinyl]-N-(tetrahydro-2H-pyran-4-yl)-benzamide (GW788388). In one specific example, the small molecule TGF-β inhibitor is 4-[4-(1,3-benzodioxol-5-yl)-5-(2-pyridinyl)-1H-imidazol-2-yl]benzamide (SB 431542).

In some examples, the TGF-β inhibitor is at a concentration of between about 0.5 to about 200 μM, or between about 1 to about 180 μM, or between about 5 to about 160 μM, or between about 10 to about 140 μM, or between about 15 to about 120 μM, or between about 20 to about 100 μM, or between about 25 to about 90 μM, or between about 30 to about 80 μM, or between about 35 to about 70 μM, or between about 40 to about 60 μM, or between about 45 to about 50 μM, or at about 1, 2, 3, 4, 5, 6, 7, 8, 9, 10, 12.5, 15, 17.5, 20, 22.5, 25, 27.5, 30, 40, 50, 60, 70, 80, 90, 100, 110, 120, 130, 140, 150, 160, 170, 180, 190 or 200 μM. In one specific example, the TGF-β inhibitor is at a concentration of 10 M.

The term “vascular endothelial growth factor” or “VEGF” as used interchangeably herein refers to a signal protein produced by cells that stimulates vasculogenesis and angiogenesis. It is part of the system that restores the oxygen supply to tissues when blood circulation is inadequate such as in hypoxic conditions. VEGF's normal function is to create new blood vessels during embryonic development, new blood vessels after injury, muscle following exercise, and new vessels (collateral circulation) to bypass blocked vessels. VEGF is a sub-family of growth factors, to be specific, the platelet-derived growth factor family of cystine-knot growth factors. They are important signalling proteins involved in both vasculogenesis (the de novo formation of the embryonic circulatory system) and angiogenesis (the growth of blood vessels from pre-existing vasculature). The VEGF family comprises in mammals five members: VEGF-A, placenta growth factor (PGF), VEGF-B, VEGF-C and VEGF-D. A number of VEGF-related proteins encoded by viruses (VEGF-E) and in the venom of some snakes (VEGF-F) have also been discovered. Activity of VEGF-A has been studied mostly on cells of the vascular endothelium, although it does have effects on a number of other cell types (e.g., stimulation monocyte/macrophage migration, neurons, cancer cells, kidney epithelial cells). In vitro, VEGF-A has been shown to stimulate endothelial cell mitogenesis and cell migration. VEGF-A is also a vasodilator and increases microvascular permeability and was originally referred to as vascular permeability factor. There are multiple isoforms of VEGF-A that result from alternative splicing of mRNA from a single, 8-exon VEGFA gene. These are classified into two groups which are referred to according to their terminal exon (exon 8) splice site: the proximal splice site (denoted VEGFₓₓₓ) or distal splice site (VEGFₓₓₓb). In addition, alternate splicing of exon 6 and 7 alters their heparin-binding affinity and amino acid number (in humans: VEGF₁₂₁, VEGF₁₂₁b, VEGF₁₄₅, VEGF₁₆₅, VEGF₁₆₅b, VEGF₁₈₉, VEGF₂₀₆; the rodent orthologs of these proteins contain one fewer amino acids). These domains have important functional consequences for the VEGF splice variants, as the terminal (exon 8) splice site determines whether the proteins are pro-angiogenic (proximal splice site, expressed during angiogenesis) or anti-angiogenic (distal splice site, expressed in normal tissues). In addition, inclusion or exclusion of exons 6 and 7 mediate interactions with heparan sulfate proteoglycans (HSPGs) and neuropilin co-receptors on the cell surface, enhancing their ability to bind and activate the VEGF receptors (VEGFRs). All members of the VEGF family stimulate cellular responses by binding to tyrosine kinase receptors (the VEGFRs) on the cell surface, causing them to dimerize and become activated through trans-phosphorylation, although to different sites, times, and extents. The VEGF receptors have an extracellular portion consisting of 7 immunoglobulin-like domains, a single transmembrane spanning region, and an intracellular portion containing a split tyrosine-kinase domain. VEGF-A binds to VEGFR-1 (Flt-1) and VEGFR-2 (KDR/Flk-1). VEGFR-2 appears to mediate almost all of the known cellular responses to VEGF. The function of VEGFR-1 is less well-defined, although it is thought to modulate VEGFR-2 signalling. Another function of VEGFR-1 may be to act as a dummy/decoy receptor, sequestering VEGF from VEGFR-2 binding (this appears to be particularly important during vasculogenesis in the embryo). VEGF-C and VEGF-D, but not VEGF-A, are ligands for a third receptor (VEGFR-3/Flt4), which mediates lymphangiogenesis. The receptor (VEGFR3) is the site of binding of main ligands (VEGFC and VEGFD), which mediates perpetual action and function of ligands on target cells. Vascular endothelial growth factor-C can stimulate lymphangiogenesis (via VEGFR3) and angiogenesis via VEGFR2. In addition to binding to VEGFRs, VEGF binds to receptor complexes consisting of both neuropilins and VEGFRs. This receptor complex has increased VEGF signalling activity in endothelial cell.

Thus, examples of VEGF that could be used in the method of the present disclosure include but are not limited to VEGF-A, VEGF-B, VEGF-C, VEGF-D and PIGF. In one specific example, the VEGF to be used is VEGF-A. Exemplary isoforms of VEGF-A include but are not limited to VEGF₁₂₁, VEGF₁₂₁b, VEGF₁₄₅, VEGF₁₆₅, VEGF₁₆₅b, VEGF₁₈₉ and VEGF₂₀₆. One specific example of the VEGF-A isoform is VEGF₁₆₅.

In some examples, the VEGF is at a concentration of between about 0.5 to about 1000 ng/ml, or between about 1 to about 900 ng/ml, or between about 5 to about 800 ng/ml, or between about 10 to about 700 ng/ml, or between about 15 to about 600 ng/ml, or between about 20 to about 500 ng/ml, or between about 25 to about 450 ng/ml, or between about 30 to about 400 ng/ml, or between about 35 to about 350 ng/ml, or between about 40 to about 300 ng/ml, or between about 45 to about 250 ng/ml, or between about 50 to about 200 ng/ml, or between about 60 to about 150 ng/ml, or between about 70 to about 100 ng/ml, or between about 80 to 90 ng/ml, or at about 1, 2, 3, 4, 5, 6, 7, 8, 9, 10, 12.5, 15, 17.5, 20, 22.5, 25, 27.5, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950 or 1000 ng/ml. In one specific example, the VEGF is at a concentration of 50 ng/ml.

In one particular example, for the first culture medium used in the method of the present disclosure, the fibroblast growth factor is fibroblast growth factor 2 (FGF2) at a concentration of 4 ng/ml, the TGF-β inhibitor is 4-[4-(1,3-benzodioxol-5-yl)-5-(2-pyridinyl)-1H-imidazol-2-yl]benzamide (SB 431542) at a concentration of 10 μM, and the VEGF is isoform VEGF₁₆₅ of VEGF-A at a concentration of 50 ng/ml.

In one example, the first culture medium used in the method of the present disclosure does not comprise an activator of the Wnt signalling pathway(s).

The term “Wnt signalling pathways” as used herein refers to a group of signal transduction pathways made of proteins that pass signals into a cell through cell surface receptors. Three Wnt signalling pathways are currently known: the canonical Wnt pathway, the noncanonical planar cell polarity pathway, and the noncanonical Wnt/calcium pathway. All three pathways are activated by binding a Wnt-protein ligand to a Frizzled family receptor, which passes the biological signal to the Dishevelled protein inside the cell. The canonical Wnt pathway leads to regulation of gene transcription. The noncanonical planar cell polarity pathway regulates the cytoskeleton that is responsible for the shape of the cell. The noncanonical Wnt/calcium pathway regulates calcium inside the cell. Wnt signalling pathways use either nearby cell-cell communication (paracrine) or same-cell communication (autocrine). They are highly evolutionarily conserved in animals.

Wnt signalling was first identified for its role in carcinogenesis, then for its function in embryonic development. The embryonic processes it controls include body axis patterning, cell fate specification, cell proliferation and cell migration. These processes are necessary for proper formation of important tissues including bone, heart and muscle. Its role in embryonic development was discovered when genetic mutations in Wnt pathway proteins produced abnormal fruit fly embryos. Wnt signalling also controls tissue regeneration in adult bone marrow, skin and intestine.

As used herein, the term “Wnt-signalling activator” refers to a molecule or compound which activates or up-regulates genes involved in the Wnt signalling pathway. In one example, the WNT-signalling activator is, but is not limited to, 2-Amino-4-[3,4-(methylenedioxy)benzylamino]-6-(3-methoxyphenyl)pyrimidine (CAS no. 853220-52-7), (1-(4-(Naphthalen-2-yl)pyrimidin-2-yl)piperidin-4-yl)methanamine (WAY 262611 or DKK1 inhibitor), WAY-316606 (5-(Phenylsulfonyl)-N-4-piperidinyl-2-(trifluoromethyl)benzene sulfonamide hydrochloride), heteroarylpyrimidines, arylpyrimidines, IQ1 (2-[2-(4-Acetylphenyl)diazenyl]-2-(3,4-dihydro-3,3-dimethyl-1(2H)-isoquinolinylidene)acetamide; CAS no. 331001-62-8), QS11 ((2S)-2-[2-(Indan-5-yloxy)-9-(1,1′-biphenyl-4-yl)methyl)-9H-purin-6-ylamino]-3-phenyl-propan-1-ol; CAS no. 944328-88-5), SB-216763 (3-(2,4-dichlorophenyl)-4-(1-methylindol-3-yl)pyrrole-2,5-dione), BIO(6-bromoindirubin-3′-oxime), deoxycholic acid (DCA), 2-amino-4-[3,4-(methylenedioxy)benzyl-amino]-6-(3-methoxyphenyl)pyrimidine, or derivatives thereof. In another example, the WNT-signalling activator is a GSK3 inhibitor. In yet another example, the GSK3 inhibitor is, but is not limited to, CHIR-99021 (6-[2-[[4-(2,4-dichlorophenyl)-5-(5-methyl-1H-imidazol-2-yl)pyrimidin-2-yl]amino]ethylamino]pyridine-3-carbonitrile), BIO(6-bromoindirubin-3′-oxime), SB 216763 (3-(2,4-dichlorophenyl)-4-(1-methylindol-3-yl)pyrrole-2,5-dione), CHIR-98014 (6-N-[2-[[4-(2,4-dichlorophenyl)-5-imidazol-1-ylpyrimidin-2-yl]amino]ethyl]-3-nitropyridine-2,6-diamine), TWS119 (3-[[6-(3-aminophenyl)-7H-pyrrolo[2,3-d]pyrimidin-4-yl]oxy]phenol), IM-12 (3-[2-(4-fluorophenyl)ethylamino]-1-methyl-4-(2-methyl-1H-indol-3-yl)pyrrole-2,5-dione), 1-azakenpaullone 9-bromo-7,12-dihydropyrido[3′,2′:2,3]azepino[4,5-b]indol-6(5H)-one, AR-A0144181-[(4-methoxyphenyl)methyl]-3-(5-nitro-1,3-thiazol-2-yl)urea, SB415286 3-(3-chloro-4-hydroxyanilino)-4-(2-nitrophenyl)pyrrole-2,5-dione, AZD1080 (3E)-3-[5-(morpholin-4-ylmethyl)-1H-pyridin-2-ylidene]-2-oxo-1H-indole-5-carbonitrile, AZD2858 3-amino-6-[4-(4-methylpiperazin-1-yl)sulfonylphenyl]-N-pyridin-3-ylpyrazine-2-carboxamide, indirubin (3E)-3-(3-oxo-1H-indol-2-ylidene)-1H-indol-2-one or derivatives thereof.

In some examples of the method of the present disclosure, culturing the endothelial lineage cells from (a) on an extracellular matrix comprises culturing the endothelial lineage cells from (a) on an extracellular matrix in contact with an endothelial growth medium. The endothelial growth medium could be any suitable endothelial growth medium that is known or commercially available. For example, EGM-2 basal medium (Lonza, cat. no. CC-3162) is used in the examples of the present disclosure.

The method of the first aspect could also be used to obtain endothelial cells from induced pluripotent stem cells (iPSCs), by deriving lateral plate mesoderm cells from induced pluripotent stem cells. Thus, in some examples, the present disclosure provides for a method of deriving and maintaining endothelial cells comprising: (a) culturing iPSC-derived lateral plate mesoderm cells under oxygen-deprived condition to obtain endothelial lineage cells; and (b) culturing the endothelial lineage cells from (a) on an extracellular matrix to maintain and expand the endothelial lineage cells.

The term “induced pluripotent stem cell” or “iPSC” as used herein refers to pluripotent stem cells that can be generated directly from adult cells. iPSCs are typically derived by introducing products of specific sets of pluripotency-associated genes, or “reprogramming factors”, into a given cell type. Upon introduction of reprogramming factors, cells begin to form colonies that resemble pluripotent stem cells, which can be isolated based on their morphology, conditions that select for their growth, or through expression of surface markers or reporter genes. In the present disclosure, the iPSCs used to generate endothelial cells include IMR90-iPSCs and BJ-iPSCs. The IMR90-iPSCs were generated from IMR90 fetal lung fibroblasts by viral transduction of a combination of OCT4, SOX2, NANOG, and LIN28. These iPSCs have a normal karyotype, demonstrate telomerase activity and express embryonic stem cell surface markers. The BJ-iPSCs were generated from BJ fibroblasts derived from neonatal foreskin tissue and adult scar tissue cells.

As used herein, the term “OCT4”, also known as octamer-binding transcription factor 4 or POU5F1 (POU domain, class 5, transcription factor 1), refers to a protein that in humans is encoded by the POU5F1 gene. OCT4 is a homeodomain transcription factor of the POU family. This protein is critically involved in the self-renewal of undifferentiated embryonic stem cells. As such, it is frequently used as a marker for undifferentiated cells. OCT4 expression must be closely regulated; too much or too little will cause differentiation of the cells.

The term “SOX2” as used herein refers to SRY (sex determining region Y)-box 2, which is a transcription factor that is essential for maintaining self-renewal, or pluripotency, of undifferentiated embryonic stem cells. SOX2 has a critical role in maintenance of embryonic and neural stem cells. SOX2 is a member of the SOX family of transcription factors, which have been shown to play key roles in many stages of mammalian development. This protein family shares highly conserved DNA binding domains known as HMG (High-mobility group) box domains containing approximately 80 amino acids.

The term “NANOG” as used herein refers to a transcription factor critically involved with self-renewal of undifferentiated embryonic stem cells. In humans, this protein is encoded by the NANOG gene.

The term “LIN28” as used herein refers to a protein that in human is encoded by the LIN28 gene. It is an RNA-binding protein that binds to and enhances the translation of the IGF-2 (insulin-like growth factor 2) mRNA. LIN28 binds to the let-7 pre-microRNA and blocks production of the mature let-7 microRNA in mouse embryonic stem cells. In pluripotent embryonal carcinoma cells, LIN28 is localized in the ribosomes, P-bodies and stress granules. LIN28 is thought to regulate the self-renewal of stem cells. In vertebrates, there are two paralogs present, LIN28A and LIN28B. In mice, LIN28 is highly expressed in mouse embryonic stem cells and during early embryogenesis. LIN28 is highly expressed in human embryonic stem cell and can enhance the efficiency of the formation of induced pluripotent stem cells (iPSCs) from human fibroblasts.

The present disclosure also provides a culture medium for deriving and maintaining endothelial cells, comprising the fibroblast growth factor, TGF-β inhibitor and VEGF, as described above. In some examples, the culture medium for deriving and maintaining endothelial cells further comprises a basal cell growth medium.

Any cell culture medium may also be supplemented with further components, as and when required based on the experiment to be performed, the cell type in questions, as well as the required status of the cell (starved or otherwise). Cell culture supplements are, but are not limited to, serum, amino acids, chemical compounds, salts, buffering salts or agents, antibiotics, antimycotics, cytokines, growth factors, hormones, lipids, and derivatives thereof.

In one example, the serum is, but is not limited to, fetal calf serum (FCS), fetal bovine serum (FBS), human serum (huS), platelet lysate (hPL), human platelet lysate (hPL), and combinations thereof.

In one example, the antimycotic is, but is not limited to, amphotericin B, clotimazol, nystatin and combinations thereof.

In one example, the amino acid is, but is not limited to, isoleucine, leucine, lysine, methionine, phenylalanine, threonine, tryptophan, valine, arginine, cysteine, cystine, histidine, tyrosine, alanine, aspartic acid, asparagine, glutamine, glutamic acid, glycine, hydroxproline, proline, serine, combinations and derivatives thereof. In one example, the amino acid is glutamine. The amino acids listed herein may be provided in either the L- or the D-stereoisomer, as required. In one example, the glutamine supplement is L-alanyl-L-glutamine dipeptide.

In a further example, the antibiotic is, but is not limited to, ampicillin, penicillin, chloramphenicol, gentamycin, kanamycin, neomycin, streptomycin, tetracycline, polymyxin B, actinomycin, bleomycin, cyclohexamide, geneticin (G148), hygromycin B, mitomycin C and combinations thereof. In one example, the antibiotic is penicillin. In another example, the antibiotic is streptomycin. In yet another example, the antibiotic is penicillin and streptomycin. In one example, the antibiotic is gentamicin.

In one example, the salt, buffering salt or agent is, but is not limited to, sodium chloride (NaCl), potassium chloride (KCl), sodium hydrogen phosphate (Na₂HPO₄), monosodium phosphate (NaH₂PO₄), monopotassium phosphate (KH₂PO₄), magnesium sulfate (MgSO₄), calcium chloride (CaCl₂), calcium chloride (CaCl₂×2H₂O), dextrose, glucose, Sodium bicarbonate (NaHCO₃) and combinations thereof.

In another example, the supplement for cell proliferation is heparin.

In one example, the growth supplement is insulin.

In another example, the growth enhancer of stem cells is laminin.

The endothelial cells obtained using the method as disclosed herein show significant expression of PECAM1. Western blot analysis demonstrated the presence of endothelial adherens junctions, CDH5, at a level that is comparable to human coronary artery endothelial cells, HCAEC. The endothelial cells obtained also formed spontaneous tube structures that stained for mature endothelial marker vWF. Further, the endothelial cells obtained possessed eNOS, and were capable of taking up acetylated LDL, resembling the mature HCAEC. Thus, the endothelial cells produced using the method as disclosed herein are fully functional endothelial cells that resemble the endothelial cells of the human coronary artery.

In a second aspect, there is provided a cell co-culture system comprising an endothelial cell culture and a hepatocyte cell culture.

The term “co-culture” as used herein refers to a cell culture setup in which two or more different populations of cells are grown with some degree of contact between them. Such “contact” could be defined by direct physical contact, i.e. where the cells of one population are in direct physical contact with the cells of another population. Direct physical contact could also be absent, which means the different cell populations are in indirect contact mediated by the sharing of culture media. The shared media will then contain metabolites generated and released by at least one population of the co-cultured cells, although it can also contain metabolites generated and released by all the different populations of co-cultured cells.

In some examples, the co-culture system comprises only two different cell populations, one being an endothelial cell culture and the other being a hepatocyte cell culture. In some other examples, the co-culture system can include additional cell population(s). In some examples, the endothelial cell culture is obtained using the method as disclosed herein or any other suitable methods. The hepatocyte cell culture can be derived from any mammalian pluripotent stem cells, for example human induced pluripotent stem cells (iPSCs), embryonic stem cells, hepatic resident stem cells (oval cells), and the like. The hepatocyte cell culture may be obtained by any suitable method, including the method described in the present application, as long as the cell culture contains metabolically active hepatocyte cells.

The term “metabolically active cells” as used herein refers to cells that are able to process substances by metabolism. Metabolically active cells generally undergo rapid cell division, contain high concentrations of mitochondria, and show high level of glucose consumption.

The hepatocytes derived from stem cells obtained from an individual donor can be cultured with the endothelial cells derived from the stem cells obtained from the same donor.

In some examples, the endothelial cell culture and the hepatocyte cell culture in the co-culture system are cultured on extracellular matrix. One specific example of a suitable extracellular matrix comprises collagen, examples of which include but are not limited to Type I, Type II, Type III, Type IV and Type V collagen. In one particular example, the extracellular matrix used comprises Type I collagen, such as that obtained from rat tail.

In some examples, the co-culture described herein is a micro-patterned co-culture, wherein a cell culture vessel comprises at least one major well which in turn comprises at least two minor wells. Each type of cell culture is seeded in each individual minor well. In one example, each minor well contains a different cell culture from its adjacent minor well. In some other examples, the co-culture is a hybrid of ECM overlay (“sandwich”) culture.

The co-culture medium used should be suitable for both the endothelial cell culture and the hepatocyte cell culture to remain as metabolically active. In some examples, the co-culture medium comprises a mixture of an endothelial cell culture medium and a hepatocyte cell culture medium. A non-limiting example of an endothelial cell culture medium is EGM-2 medium, and a non-limiting example of a hepatocyte cell culture medium is William's E medium. In one example, both cell culture media do not contain serum. In another example, both cell culture media contain serum. In some other examples, at least one cell culture medium contains serum. The ratio of the two cell culture media depends on the number of cells of each cell population, and can be in the range of about 2:1, or about 1.9:1, or about 1.8:1, or about 1.7:1, or about 1:6:1, or about 1.5:1, or about 1.4:1, or about 1.3:1, or about 1.2:1, or about 1.1:1, or about 1:1, or about 1:1.1, or about 1:1.2, or about 1:1.3, or about 1:1.4, or about 1:1.5, or about 1:1.6, or about 1:1.7, or about 1:1,8, or about 1:1.9, or about 1:2. In one specific example, the co-culture medium comprises a mixture of an endothelial cell culture medium and a hepatocyte cell culture medium in the ratio of 1:1.

The cell co-culture system of the present disclosure can be further developed into other platforms. For example, cells-on-chip in microfluidic-based systems could provide the benefits of different flow dynamics, minimizing reagents used, and optical suitability for high-content imaging of cells. Thus, in a third aspect, there is provided a microfluidic-based system comprising the cell co-culture system of the present invention.

The term “microfluidic” or “microfluidics” as used herein refers to the science and technology of manipulating and controlling fluids, usually in the range of microliters to picoliters, in networks of channels with lowest dimensions from tens to hundreds micrometers. Microfluidic structures include micropneumatic systems, i.e. microsystems for the handling of off-chip fluids (liquid pumps, gas valves, etc.), and microfluidic structures for the on-chip handling of nanoliter and picoliter volumes.

The co-culture system or the microfluidic-based system as disclosed herein can be used for disease modeling or drug testing. Thus, in a fourth aspect, there is provided a method of disease modeling or drug testing using the cell co-culture system or the microfluidic-based system of the present invention.

In some examples, the method of disease modeling or drug testing as described herein utilizes the metabolic activities of the hepatocytes in the co-culture. For example, the method can be carried out to test the effect of a candidate compound on suppressing the inflammatory response of the endothelial cells. The candidate compound will first be metabolized by the hepatocytes in the co-culture, and the metabolites produced by the hepatocytes will then be released into the co-culture medium. The effects of the released metabolites on the endothelial cells will then be assessed. Such a method mimics the process by which a candidate compound is metabolized in the human body, thus serving as a replacement model for in vivo drug testing.

As described above, since the endothelial cells obtained using the methods as disclosed herein resemble the functional mature endothelial cells of the human coronary artery, the co-culture system comprising the endothelial cells obtained using the methods as disclosed herein could be used for coronary artery disease modeling or screening of candidate drug or therapeutic agent for the treatment of coronary artery diseases or disorders.

The inventors of the present application also demonstrated that hPSC derived endothelial cells could in turn impact the metabolic function of the hepatocytes derived from hPSCs. For example, there was significant increase of CYP gene expressions in co-cultured hPSC-HEPs, suggesting that the presence of hPSC-ECs could promote metabolic activity in hPSC-HEPs. Therefore, a co-culture of hPSC-ECs and hPSC-HEPs could better recapitulate the in vivo vascular-liver systemic interactome. Thus, the co-culture system or the microfluidic-based system as disclosed herein could also be used to develop and screen drug candidates for treating hepatic diseases or disorders, or for screening the hepatotoxicity of drug candidates for treating any other diseases or disorders, in particular vascular diseases or disorders. For example, following contact of the co-culture with a candidate therapeutic agent, various cellular functions in the hepatocytes may be assessed by examining gene expression, albumin production, urea production cytochrome P450 (CYP) metabolic activity or any inducible liver enzyme activity, uptake and secretion of liver-specific products, and response to hepatotoxins, by detecting and/or measuring level of a protein, metabolite, reporter molecule, label, or gene expression level such as through gene fluorescence in the cell or in the culture medium.

In one example, the disease modeling comprises modeling of inflammation or drug-induced vascular injury. In some examples, the drug testing comprises testing the effect of the drug on the endothelial cell culture, testing drug-induced vascular injury, testing hepatotoxicity, predicting vascular protection, predicting drug efficacy or predicting drug safety.

Some non-limiting examples of a candidate drug or therapeutic agent include a small molecule, a peptide, a polypeptide, an antibody, an oligonucleotide or a polynucleotide. In one specific example, the candidate therapeutic agent is a nutraceutical.

The term “nutraceutical” as used herein refers to a pharmaceutical-grade and standardized nutrient. They are products derived from food sources that are purported to provide extra health benefits, in addition to the basic nutritional value found in foods. Products may be useful in preventing chronic diseases, improving health, delaying the aging process, increasing life expectancy, or supporting the structure or function of the body. Major classes of nutraceuticals include but are not limited to dietary supplements and functional foods. A dietary supplement is a product that contains nutrients derived from food products that are concentrated in liquid or capsule form. It is a product taken by mouth that contains a “dietary ingredient” intended to supplement the diet. The “dietary ingredients” in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders. Functional foods are designed to allow consumers to eat enriched foods close to their natural state, rather than by taking dietary supplements manufactured in liquid or capsule form. Functional foods have been either enriched or fortified, a process called nutrification. This practice restores the nutrient content in a food back to similar levels from before the food was processed. Sometimes, additional complementary nutrients are added, such as vitamin D to milk. Functional foods can be defined as ordinary food that has components or ingredients added to give it a specific medical or physiological benefit, other than a purely nutritional effect. Some specific non-limiting examples of nutraceuticals include: quercetin and genistein. Quercetin, a naturally occurring flavonoid compound, is found commonly in food, such as tea, onions, berries and apples. It exerts various beneficial effects through its anti-inflammatory and anti-oxidant properties. Quercetin intake is also correlated with lower incidence of coronary heart disease and stroke. Genistein, a potent phytoestrogen, is effective in mitigating endothelial dysfunction and exerts an anti-inflammatory effect by down-regulating the NFκB pathway.

In some examples, the nutraceuticals have to be processed to their metabolites, which could then exert their therapeutic effects on the target cells. For example, as demonstrated in the present application, nutraceuticals quercetin and genistein as administered in their natural occurring state do not show significant anti-inflammatory effects on hPSC-derived endothelial cells. However, after being metabolized by hepatocytes in the hepatocytes-endothelial cells co-culture, quercetin and genistein have been bio-activated, which in turn exerted significant anti-inflammatory effects on the endothelial cells in the same co-culture.

The co-culture can be exposed to varying concentrations of the candidate drug or therapeutic agent, which could be determined according to the knowledge available to a person skilled in the art, an amount representing a proposed dose or range or proposed doses in a clinical setting.

In some examples, the stem cell derived endothelial cells may be derived from stem cells donated from one or more subjects suffering from a vascular disease or disorder, and the methods encompass preparing endothelial cells using such stem cells, and preserving the phenotype of such endothelial cells. Likewise, in some other examples, the stem cell derived hepatocytes may be derived from stem cells donated from one or more subjects suffering from a disease or disorder of the liver, and the methods encompass preparing hepatocytes using such stem cells, and preserving the phenotype of such hepatocytes. Maintenance of the diseased or disordered phenotype may be identified using any of the methods known in the art.

The dwell time, or the time over which the hepatocytes in the co-culture are exposed to the candidate drug or therapeutic agent may be, according to knowledge available to those skilled in the art, a period of hours, days, weeks or months representing time course of exposure in a clinical setting. In some examples, the dwell time is about 6 hours to about 4 days, or about 12 hours to about 3 days, or about 24 hours to about 2 days, or at about 6 hours, about 9 hours, about 12 hours, about 18 hours, about 24 hours, about 36 hours, about 48 hours, about 60 hours, about 72 hours, about 84 hours or about 96 hours. In one specific example, the dwell time is about 48 hours. After incubation with the candidate drug or therapeutic agent, the cells in the co-culture are examined to determine impact of the agent or the metabolites of the agent on the hepatocytes or the endothelial cells or both. Thus, in some examples, the drug testing comprises (a) co-culturing the endothelial cell culture and the hepatocyte cell culture with the drug for 6 hours to 4 days, or 12 hours to 3 days, or 24 hours to 2 days, or at 6 hours, 9 hours, 12 hours, 18 hours, 24 hours, 36 hours, 48 hours, 60 hours, 72 hours, 84 hours or 96 hours to obtain hepatocyte metabolized drug; and (b) measuring the effect of the hepatocyte metabolized drug from (a) on the endothelial cell culture.

The co-culture system can also be used to determine the therapeutic effects or hepatotoxicity arising from drug interactions. For example, the potential therapeutic effect on the endothelial cells of an interaction between a first test compound and a second test compound can be examined by contacting a co-culture as described herein with the first and second test compounds. The co-culture is then maintained for a time and under conditions sufficient to allow an effect of an interaction between the first and second test compounds on the stem cell-derived endothelial cells, and taking a test measurement and/or otherwise obtaining test data as described herein, which is indicative of the effect of the interaction of the first and second test compounds on the endothelial cells. Likewise, the hepatotoxicity of an interaction between a first and a second test compounds can be determined accordingly.

As used herein, the singular form “a”, “an” and “the” include plural references unless the context clearly dictates otherwise. For example, the term “a primer” includes a plurality of primers, including mixtures thereof.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value. When used in the context of duration of time, the term “about” typically means +/−20% of the stated time, more typically +/−15% of the stated time, more typically +/−10% of the stated time, more typically, +/−5% of the stated time, even more typically +/−2% of the stated time, and even more typically +/−1% of the stated time. For example, when the stated duration of time is 1 day, the term “about 1 day” could refer to 1 day +/−0 to 6 hours. As another example, when the stated duration of time is 1 hour, the term “about 1 hour” could refer to 1 hour +/−0 to 10 minutes.

Throughout this disclosure, certain examples may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

The following examples illustrate methods by which aspects of the invention may be practiced or materials suitable for practice of certain embodiments of the invention may be prepared.

### Example 1—Materials and Methods

hPSC Culture and Maintenance

hPSCs were passaged using gentle cell dissociation agent (Stemcell Technologies, cat. no. 07174) and seeded onto matrigel-coated plate in mTeSR1 media (Stemcell Technologies, cat. no. 05850). H9-ESC and IMR90-iPSC were bought from WiCell Research Institute. BJ-iPSC was kindly provided by a collaborator's lab.

Generation of Endothelial Cells from hPSCs

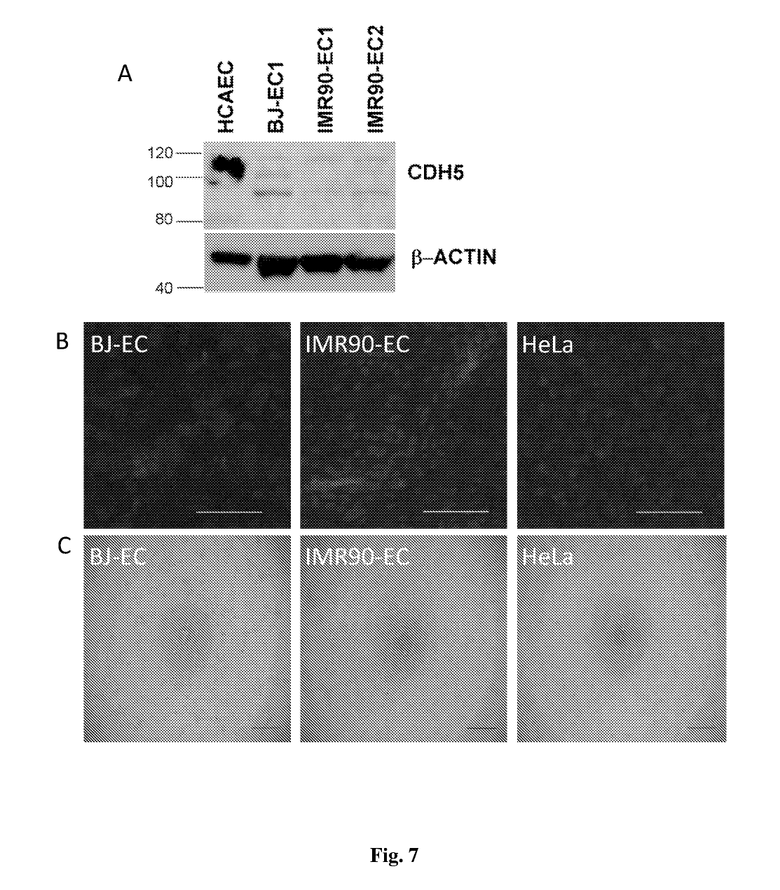

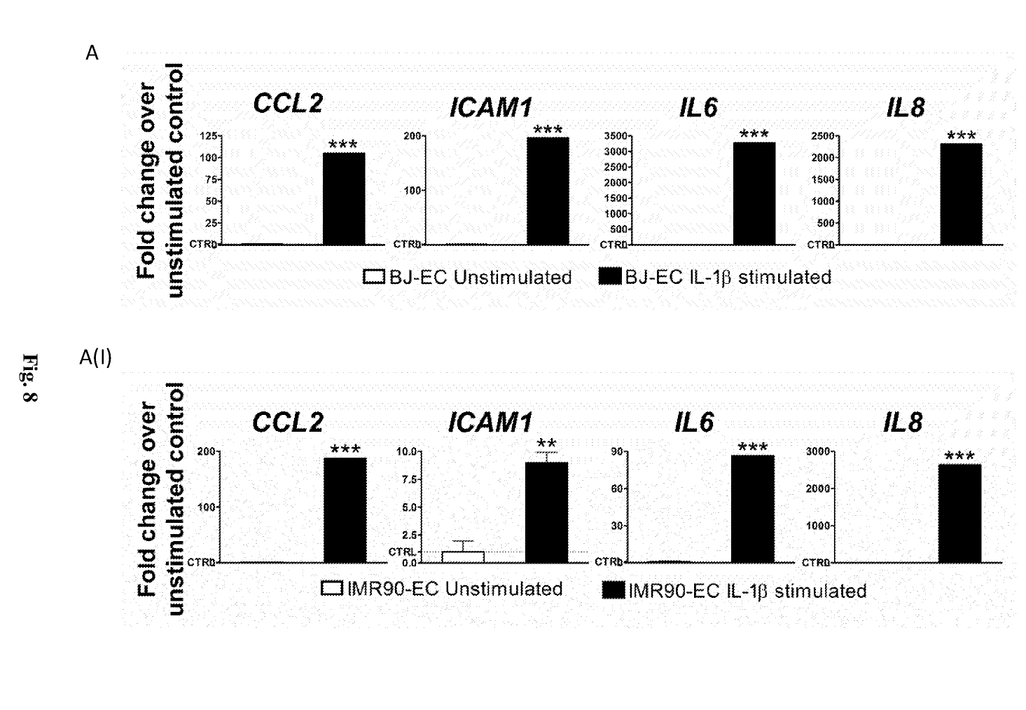

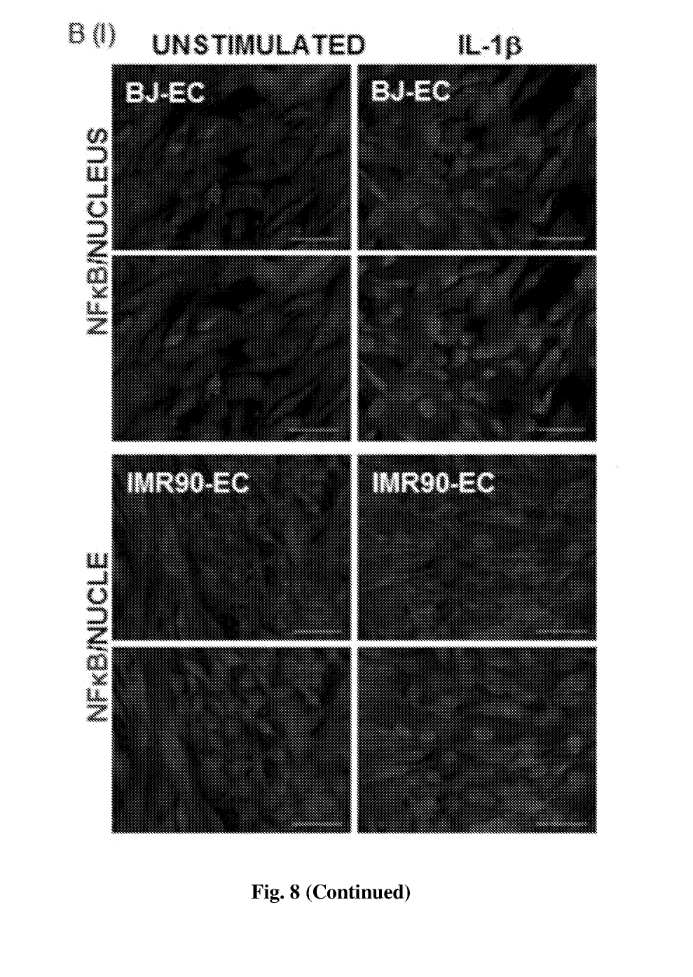

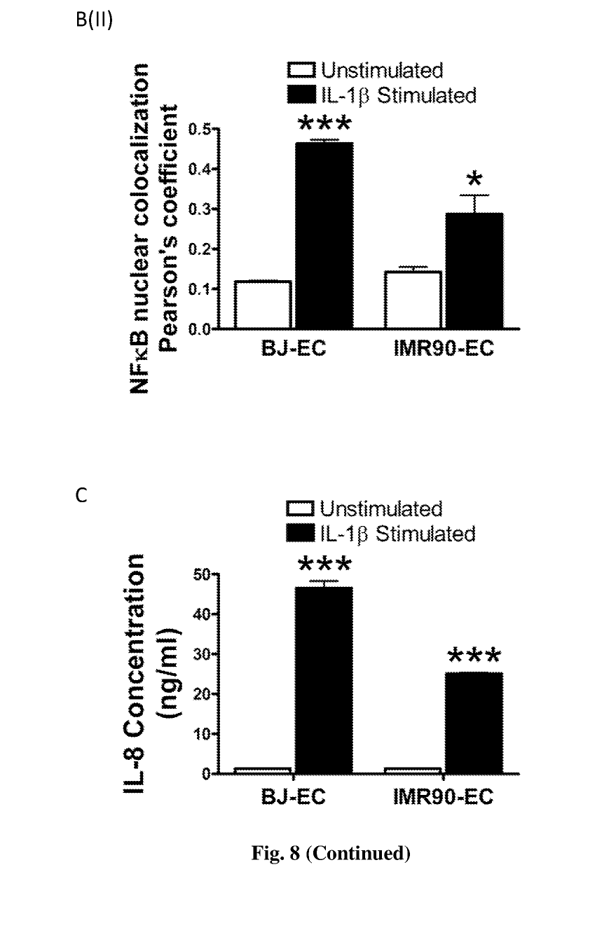

After the hPSC colonies attached, they were first induced to drive early mesoderm differentiation for 36 hours, using a chemically defined media, supplemented with human recombinant FGF2 (20 ng/ml, R&D Systems, cat. no. 233-FB), LY294002 (10 μM, Sigma, cat. no. L9908), and human recombinant BMP4 (10 ng/ml, R&D Systems, cat. no. 314-BP). Lateral plate mesoderm was further induced for another 3.5 days in media supplemented with human recombinant FGF2 (20 ng/ml) and BMP4 (50 ng/ml), with media change every 2 days. On day 5, the lateral plate mesoderm population was trypsinized using TrypLE Express (Gibco, cat. no. 12604) and plated on matrigel-coated plates in the same basal media supplemented with FGF2 (4 ng/ml), SB431542 (10 μM, Sigma, cat. no. S4317) and VEGF (50 ng/ml, R&D Systems, cat. no. 293-VE) with media change every 2-3 days. On day 10, PECAM1-expressing endothelial cells were sorted using fluorescence-activated cell sorting with PECAM1 antibody (Biolegend, cat. no. 303110). The PECAM1+ cells were plated at a density of 5×10⁴/cm² onto collagen I-coated plate in commercial media EGM-2 (Lonza, cat. no. CC-3162). For every 500 ml EGM-2 media used, the following supplements are included: human epidermal growth factor (0.5 ml); VEGF, 0.5 ml; R3-insulin growth factor-1, 0.5 ml; Ascorbic acid, 0.5 ml; Hydrocortisone, 0.2 ml; hFibroblast growth factor-beta, 2.0 ml; FBS, 10.0 ml; Gentamicin Amphotericin, 0.5 ml. hPSC-ECs were passaged using TrypLE Express when they reached more than 75% confluency. Fresh complete EGM-2 media were replaced every 2-3 days. All experiments were performed on cells up to passage 10. hPSC-ECs derived from three different cell lines, H9-ESCs (FIGS. 1-5), IMR90-iPSCs and BJ-iPSCs (FIGS. 7-8), were functionally characterized and used in inflammatory activation assays. In EC marker and functional characterization, human coronary artery ECs (HCAECs) were used as positive control, while HeLa cells and human hepatocellular carcinoma cells (HUH7) were used as negative controls.

Generation of Hepatocytes from hPSCs

Hepatocytes were generated from hPSCs by a growth factor-based differentiation protocol described in our previous protocol [1]. After 20 days of differentiation, the cells were harvested using a serial 2× TrypLE Express treatment and further dissociated into single cells by passing them through a 40 μm cell strainer. These single cells were then seeded at 2.5×10⁵ cells per well in a Collagen I (50 μg/ml, Biolaboratories, Cat. No.: 354236)-coated dishes. Attachment and recovery were promoted by seeding them in step IV differentiation medium with Hepatocyte growth factor (R&D, Cat. No.: 294-HGN-005), Follistatin (R&D, Cat. No.: FS-288), Oncostatin (R&D, Cat. No.: 295-OM-010) and Y-27632 (Rock Inhibitor) to prevent anoikis in the freshly harvested hPSC-HEPs. Next day, media was changed to Williams E medium (Sigma, Cat. No.: W1878) without serum and cells were serum starved overnight prior to nutraceutical treatments. Nutraceuticals quercetin (Sigma, Cat. No.: Q4951) and genistein (Sigma, Cat. No.: G6649) were administered at a single dose of 10 μM. Hepatocytes in this work were derived from H9-ESCs. In hepatic characterization, primary human hepatocytes (PHH) and HUH7 cells were used as positive controls, while HeLa cells were used as negative control.

HPSC-HEP Conditioned Media Experiments on hPSC-ECs

HPSC-HEPs (1.25×10⁵ cells/cm²) were cultured for 48 hour in 1:1 William's E medium+EGM-2 without serum containing 10 μM Quercetin or 10 μM Genistein. Conditioned media from hPSC-HEPs treated with quercetin or genistein were collected, and added to hPSC-ECs (that were serum starved overnight) along with 20 ng/ml of IL-13 for determination of NFκB nuclear colocalization (at 1 hour), gene expression profile of inflammatory markers (at 6 hour), and IL-8 protein levels (1 day) upon inflammation.

Endothelial-Hepatocyte Co-Culture Experiments on IBIDI 2×9 Wells

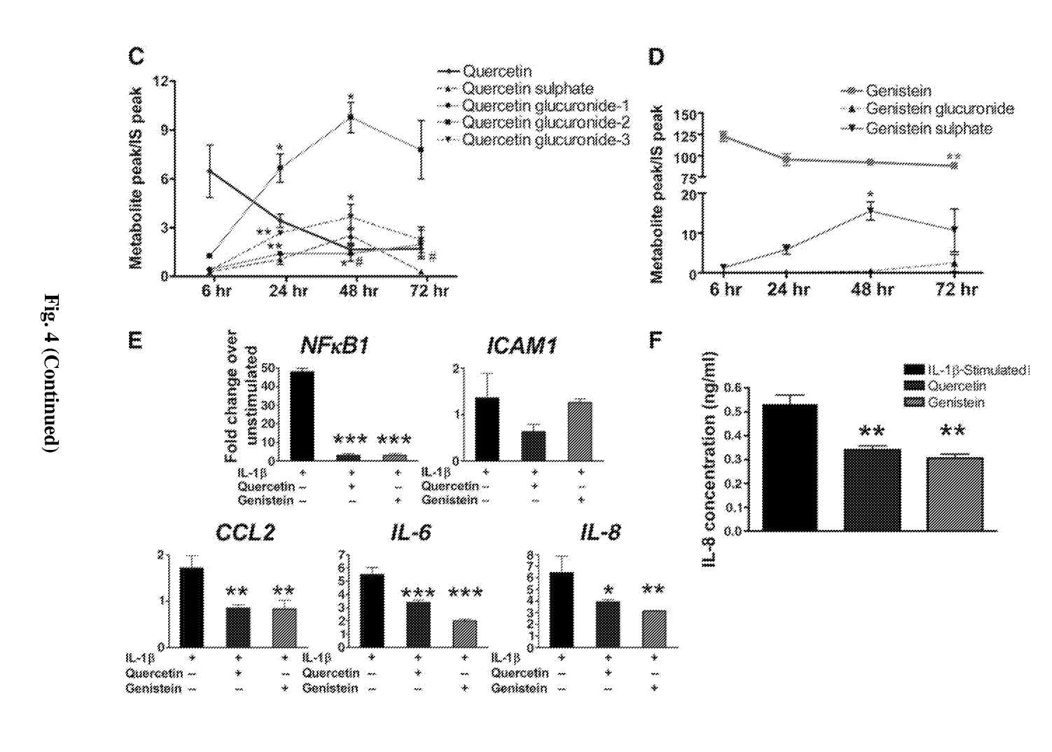

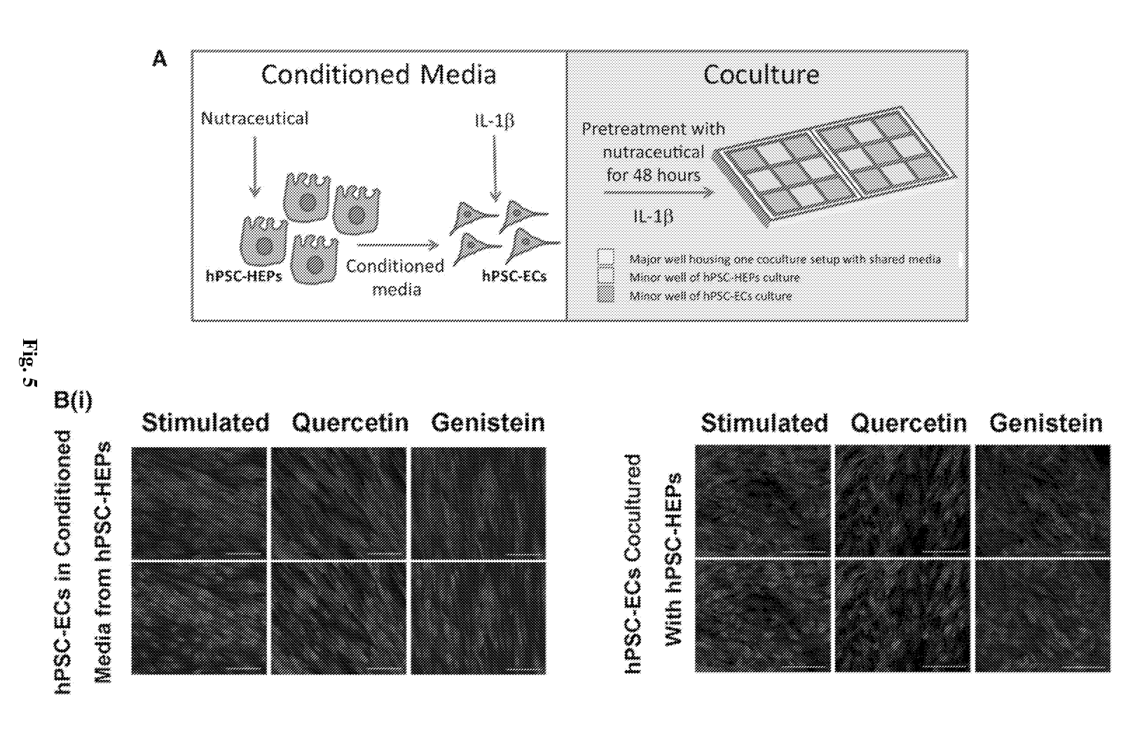

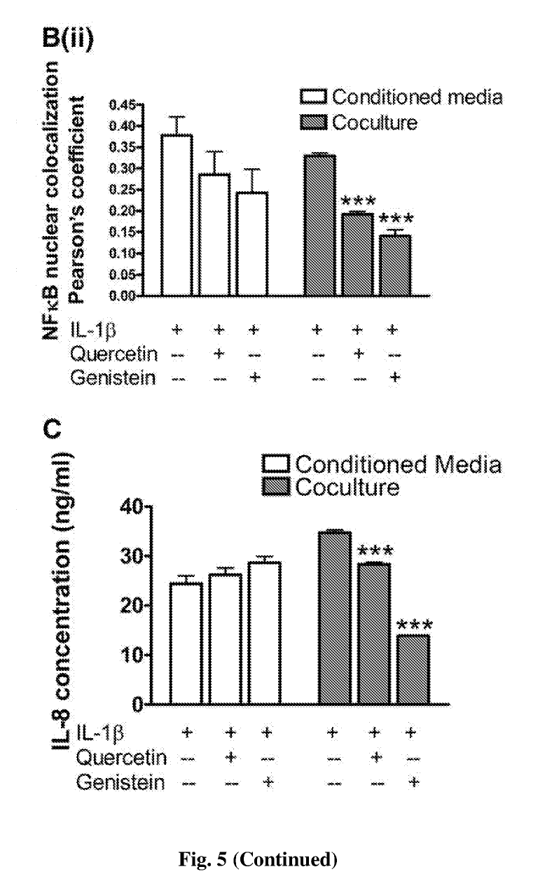

H9-ESCs were used to generate the ECs and hepatocytes for co-culture experiments and assays with nutraceuticals. Both cell types were derived from an isogenic source of hPSC to ensure a robust endothelial-hepatic model without confounding phenotypic differences due to genetic variations. The IBIDI μ-slide 2×9 wells were used for creating the co-culture setup. Each minor well was first coated with Collagen I and then a total of 2.5×10⁵ hPSC-ECs and hPSC-HEPs were seeded in individual wells in a 1:1 ratio within a major well to allow equivalent contribution of paracrine factors between the two cell types (FIG. 5A). After cell attachment, the media was changed to 1:1 William's E and EGM-2 without serum, to be commonly shared by hPSC-ECs and hPSC-HEPs in a major well. Upon overnight serum starvation, the co-cultures were pre-conditioned with 10 μM of Quercetin or Genistein for 48 hours prior to IL-1β stimulation.

Real Time Quantitative Polymerase Chain Reaction

Total RNA was prepared using RNeasy mini kit (Qiagen, cat. no. 74104). Each RNA sample (250 ng) was reverse transcribed into cDNA using Maxima First Strand cDNA Synthesis kit (Thermo, cat. no. K1641). qPCR was performed on a StepOnePlus Real-Time PCR system (Thermo) using FAST SYBR green master mix (Thermo, cat. no. 4385616). Expression levels were normalized to the housekeeping gene glyceraldehyde 3-phosphate dehydrogenase (GAPDH). The primer sequences are listed in Table 1.

TABLE 1
Primer sequences for qPCR

| Genes | Sequences | SEQ ID No.: |
|---|---|---|
| CD31 | CAGGCGCCGGGAGAAGTGAC | 1 |
| | CGTCCAGTCCGGCAGGCTCT | 2 |
| CDH5 | TGGCCAGCTGGTCCTGCAGAT | 3 |
| | TGCCCGTGCGACTTGGCATC | 4 |
| TIE2 | GCAGTGCAATGAAGCATGCCACC | 5 |
| | GGTAGCGGCCAGCCAGAAGC | 6 |
| CD34 | CACAGGAGAAAGGCTGGGCGA | 7 |
| | TGGCCGTTTCTGGAGGTGGC | 8 |
| VWF | TAGCCCGCCTCCGCCAGAAT | 9 |
| | CCTGCAGGCGCAGGTGAAGT | 10 |
| VEGFα | GCACATAGGAGAGATGAGCTTC | 11 |
| | CCACAGGGACGGGATTTCTTG | 12 |
| NFKB1 | ACTACCTGGTGCCTCTAGTGA | 13 |
| | TTTGACCTGAGGGTAAGACTTCT | 14 |
| MCP1 | AAGCTCGCACTCTCGCCTCCA | 15 |
| | GCATTGATTGCATCTGGCTGAGCG | 16 |
| IL6 | AAATTCGGTACATCCTCGACGG | 17 |
| | GGAAGGTTCAGGTTGTTTTCTGC | 18 |
| IL8 | TTGGCAGCCTTCCTGATTTCTGCAG | 19 |
| | ACAACCCTCTGCACCCAGTTTTC | 20 |
| VCAM1 | ATGGTCGCGATCTTCGGAGCC | 21 |
| | AACGGACTTGGCCCCCTCTGT | 22 |
| ICAM1 | ACCGGAAGGTGTATGAACTGA | 23 |
| | TGGTTGGCTATCTTCTTGCAC | 24 |
| bFGF | AGCGACCCTCACATCAAGCTACA | 25 |
| | CTGCCCAGTTCGTTTCAGTGCCA | 26 |
| CYP3A4 | AAGTCGCCTCGAAGATACACA | 27 |
| | AAGGAGAGAACACTGCTCGTG | 28 |
| CYP3A7 | TGCTTTGTCCTTCCGTAAGGG | 29 |
| | CAGCATAGGCTGTTGACAGTC | 30 |
| CYP2B6 | GCACTCCTCACAGGACTCTTG | 31 |
| | CCCAGGTGTACCGTGAAGAC | 32 |
| CYP1A1 | ACATGCTGACCCTGGGAAAG | 33 |
| | GGTGTGGAGCCAATTCGGAT | 34 |
| CYP1A2 | ATGCTCAGCCTCGTGAAGAAC | 35 |
| | GTTAGGCAGGTAGCGAAGGAT | 36 |
| CYP2C9 | GCCTGAAACCCATAGTGGTG | 37 |
| | GGGGCTGCTCAAAATCTTGATG | 38 |

Flow Cytometry

Cells were trypsinized and stained using 5 μl of anti-human CD31 antibody (Biolegend, cat. no. 303110) diluted in 80 μl of phosphate buffered saline (PBS) with 20% FBS per 10⁶ cells, for 1 hour at 4° C. After which cold PBS was used to wash the cells. The cell pellet was collected by centrifugation at 200 g for 3 min. The cell pellet was resuspended in 350 μl of PBS with 20% FBS for sorting. PECAM1+ cells were sorted using FACSAria IIu SORP cell sorter (BD Biosciences) and collected in PBS containing 20% FBS.

Immunocytochemistry

Cells were fixed with 4% paraformaldehyde (Nacalai Tesque, cat. no. 09154-14) and permeabilized by 0.5% Triton X-100 (Acros Organics, cat. no. 215680010) in PBS with Ca²⁺ and MG²⁺ at room temperature. Blocking was performed using PBS with 10% serum overnight at 4° C. Cells were incubated with the respective primary antibody diluted in 0.1% serum containing PBS for 1 hour and secondary antibodies in 0.1% serum containing PBS for another hour. Washes were performed twice using 0.1% serum containing PBS. DAPI (1 μg/ml, Thermo, cat. no. D3571) was used to stain the cell nucleus for 10 min. The primary antibodies used are listed in Table 2.

TABLE 2
List of primary antibodies used

| | | Dilution | |
|---|---|---|---|
| Proteins | Applications | factors | Source |
| NFκB | IMMUNO | 1:200 | ABCAM (AB32536) |
| VWF | IMMUNO | 1:100 | ABCAM (AB9378) |
| CDH5 (VE-cadherin) | WESTERN | 1:1000 | ABCAM (AB33168) |
| B-ACTIN | WESTERN | 1:1000 | SIGMA (SAB2100037) |
| eNOS | IMMUNO | 1:100 | ABCAM (AB5589) |
| Albumin | IMMUNO | 1:100 | R&D (MAB1455-SP) |

Tube Formation Assay

Matrigel-coated plates for tube formation were prepared by adding 50 μl of matrigel (10 mg/ml, BD, cat. no. 356234) per well of 96-well plate and incubation at 37° C. for 30 min. Cells were trypsinized and plated at a density of 9.3×10⁴ cells/cm² in 150 μl of complete EGM-2 per well. Images were taken hourly using inverted microscope (Olympus CKX41) at 5× magnification. Quantitative analysis of tube characteristics was performed by WimTube image processing online software (www.wimasis.com).

Acetylated LDL Uptake Assay

DiO-Ac-LDL (Biomedical Technologies, cat no. BT-925) was diluted in complete EGM-2 at 10 μg/ml before incubation with hPSC-ECs at 37° C. with 5% CO₂ for 4 hours. The cells were visualized and imaged using Olympus fluoview inverted confocal microscope at 20× magnification.

Enzyme-Linked Immunosorbent Assay

Conditioned EGM-2 media were collected, and concentration of human IL-8 was determined using the human IL-8 ELISA kit (Invitrogen, cat. no. KHC0081) according to manufacturer's instructions.

Western Blot

The cell lysates were collected using radio immunoprecipitation assay buffer (RIPA) (Thermo, cat. no. 89901) containing 1× proteinase inhibitor cocktail (Sigma, cat. no. P8340). Protein quantification was done using Quant-iT protein assay kit (Thermo, cat. no. Q32210). 80 μg of cell lysates were separated by NuPAGE 10% Bis-Tris Gel (Thermo, cat. no. NP0303BOX) and transferred onto a nitrocellulose membrane. MagicMark XP western protein standard (Thermo, cat. no. LC5602) was used to determine the molecular weight of protein bands. WesternDot 625 goat anti-rabbit western blot kit (Thermo, cat. no. W10142) was used to visualize the protein bands. Blocking was done at 4° C. overnight using 3% skimmed milk in 1× wash buffer provided by the kit and stained with CDH5 antibody in 3% skimmed milk solution for 1 hr at room temperature. The protein bands were visualized and imaged using Bio-Rad ChemiDoc MP Imaging System.

LC-MS

The metabolic potential of the hPSC-HEPs and primary rat hepatocytes (freshly isolated according to our previously established protocol [2]) were tested by exposing them to nutraceuticals quercetin and genistein (10 μM) over different durations. Internal standard (Emodin, 10 ng/ml) was added to the conditioned media. The SPE column (Phenomenex, Strata C18-E, 55 μm, 70 Å) was conditioned by washing with 1 ml methanol and then 2 ml DI water. Conditioned media was added into the column and 1.5 mL 30% methanol was added to elute the impurity such as phenol red in the medium. 0.1% Formic acid methanol was added to the column to elute all the metabolites and internal standard out to a 15 mL tube. Liquid in the 15 mL tube was dried under N₂ in a sample concentrator with 30° C. heater. After drying the sample, 100 μL of 0.1% formic acid methanol was added to the 15 mL tube and vortex for 30 s and transfer to another 1.5 mL tube. The samples were then centrifuged at 13,000 rpm for 10 min at 4° C. and 10 μl of the supernatant was injected into LC-MS. High performance liquid chromatography combined with electrospray ionization ion trap time-of-flight multistage mass spectrometry (HPLC-DAD-ESI-ITTOF-MSn) analyses were performed with a Shimadzu LCMS-IT-TOF instrument, which was composed of two LC-20AD pumps, an SIL-20AC autosampler, a CTO-20A column oven, a CBM-20A system controller, an ESI ion source, and an IT-TOF mass spectrometer (Shimadzu, Kyoto, Japan).

Statistical Analysis

Data were expressed as mean±standard deviation (SD) of at least three biological replicates of independent experiments. Statistical comparisons were conducted by Student's unpaired T-test with 95% confidence interval for two groups of samples or one-way ANOVA with Bonferroni's post-hoc test in multiple group comparisons. Analyses were carried out with Graphpad Prism 5 software.

### Example 2—Derivation of Functional Endothelial Cells from Human Pluripotent Stem Cells

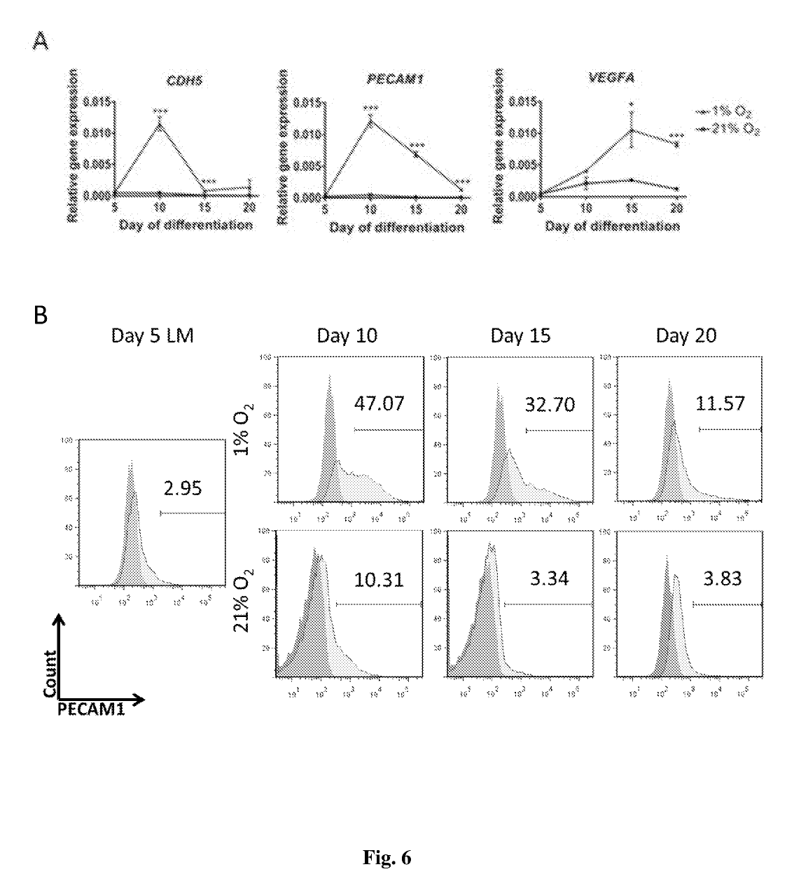

Lateral plate mesoderm is a precursor tissue of vascular lineages. Fibroblast growth factor 2 (FGF2), bone morphogenetic protein 4 (BMP4) and phosphoinositide 3-kinase inhibitor (LY294002) were used to induce lateral plate mesoderm for 5 days (FIG. 1A). A combination of factors was used to drive endothelial specification. Transforming growth factor beta (TGF-β) inhibition using small molecule SB431542 enhances endothelial differentiation of hPSCs, possibly by counteracting growth of mural cells which could arise from a common cardiovascular progenitor. FGF2 and vascular endothelial growth factor (VEGF) are mitogens for promoting angiogenesis and endothelial development. Hypoxic conditions increase efficiency of endothelial differentiation, as up-regulation of hypoxia-inducible factor triggers downstream targets that play an important role in early blood vessel development. To induce endothelial differentiation, the day 5 mesodermal population was dissociated and plated down as single cells. These cells were cultured under 1% oxygen (O₂) in a chemically defined medium containing SB431542, FGF2 and VEGF (FIG. 1A). Endothelial genes were significantly enhanced in 1% O₂ as compared to 21% O₂, peaking primarily around day 10 (FIG. 6A). Flow cytometric analysis further supported that endothelial specification was optimal around day 10 in 1% O₂, with more than 45% of the cells positive for PECAM1 (FIG. 1B, FIG. 6B). This protocol generated a sufficient yield of PECAM1+ cells for cell sorting on day 10 of differentiation, giving rise to a purity of 98.43±0.16% (FIG. 1C). This PECAM1+ population was then grown on collagen I coating and expanded using a commercial endothelial growth media (EGM-2). These cells are referred to as hPSC-ECs.

Western blot demonstrated the presence of endothelial adherens junctions, CDH5, in hPSC-ECs and the positive control, human coronary artery endothelial cells, HCAEC (FIG. 1D). Different glycosylated forms of CDH5 were found in hPSC-ECs, close to the molecular weights of those in HCAEC. The inventors postulated that there might be differences in glycosaminoglycan synthesis enzymes in hPSC-ECs and HCAEC. The hPSC-ECs formed spontaneous tube structures that stained for the mature endothelial marker, von Willebrand factor (vWF) (FIG. 1E). In addition, hPSC-ECs possessed endothelial nitric oxide synthase (eNOS) (FIG. 1F), and were capable of taking up acetylated low density lipoprotein (LDL) (FIG. 1G), resembling HCAEC but not the negative controls. The inventors observed comparable tube forming capability between hPSC-ECs and HCAEC (FIG. 1H). This endothelial differentiation protocol was reproduced on two other hPSC lines, namely the BJ and IMR90 induced pluripotent stem cells. The BJ- and IMR90-derived ECs also expressed endothelial proteins (FIG. 7A, B), as well as demonstrated tube formation capability (FIG. 7C). These functional hPSC-ECs were subsequently used for assay development.

### Example 3—HPSC-Derived Endothelial Cells Respond to Inflammatory Stimulation

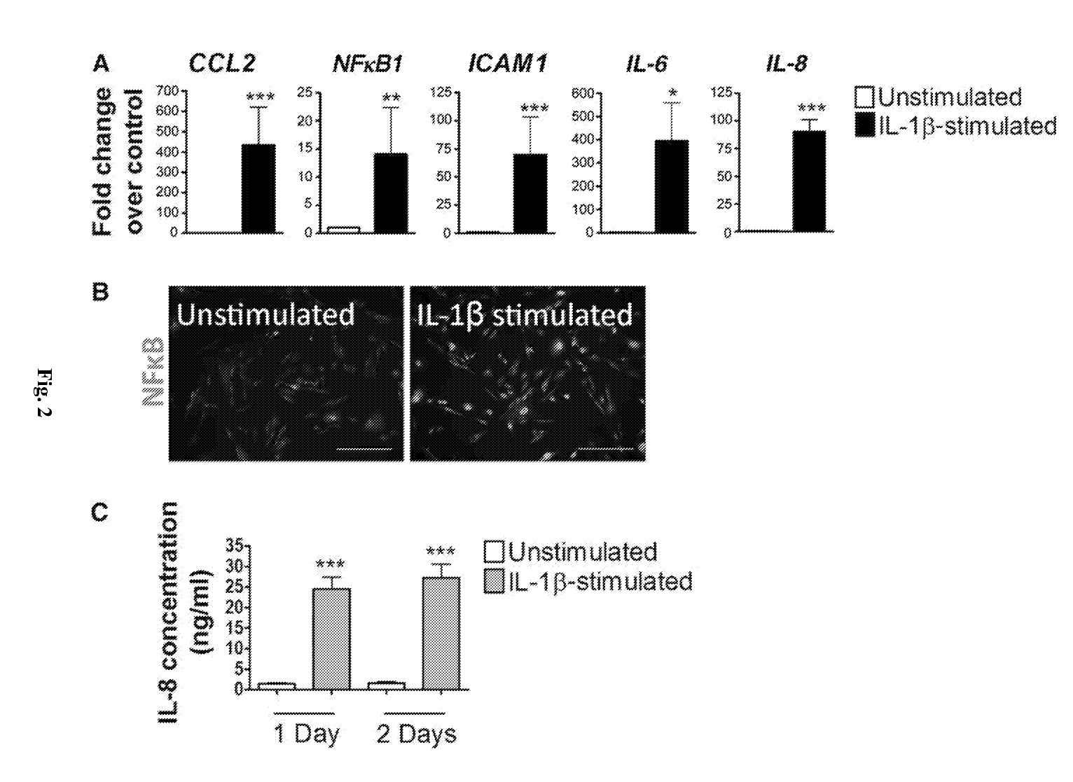

Inflammation is a hallmark of atherosclerosis. To recapitulate atherosclerosis-associated phenotypes in hPSC-ECs, an inflammatory cytokine, interleukin-1 beta (IL-1β) which is widely implicated in atherosclerosis, was used. Upon stimulation with human recombinant IL-1β, hPSC-ECs responded with a significant up-regulation of inflammatory genes (FIG. 2A). Nuclear translocation of nuclear factor kappa B (NFκB), activating major pro-inflammatory mediators, has been observed in human atherosclerotic lesions. Likewise, nuclear translocation of NFκB was evident in hPSC-ECs after stimulation with IL-1β (FIG. 2B). The production of interleukin 8 (IL-8) from conditioned media of IL-1β-stimulated hPSC-ECs was significantly higher than that of the unstimulated cells (FIG. 2C). Besides H9-ECs, the inventors also validated that BJ-ECs and IMR90-ECs could respond to IL-1β by up-regulation of inflammatory genes, increase of NFκB nuclear translocation, as well as elevated IL-8 production (FIG. 8). Hence, the inventors were able to monitor hPSC-EC inflammatory activation using a range of phenotypic readouts.

### Example 4—Nutraceuticals are not Effective in Suppressing the Inflammatory Response of hPSC-Derived Endothelial Cells

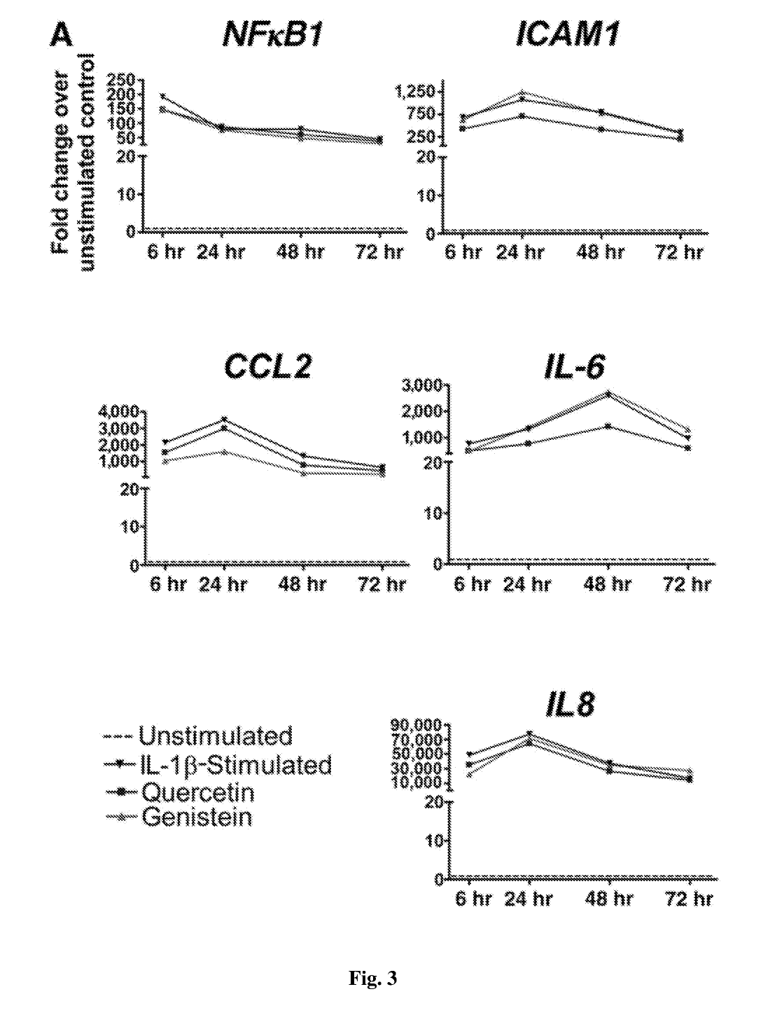

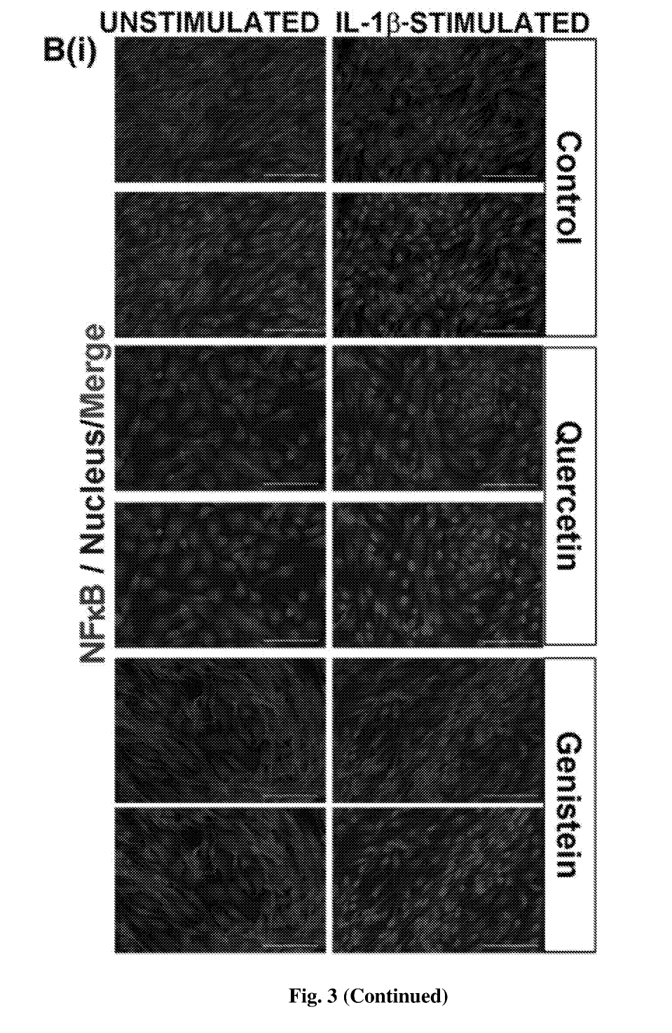

Next, the inventors tested whether administration of nutraceuticals quercetin and genistein could suppress inflammatory responses in IL-1β-stimulated hPSC-ECs. Quercetin, a naturally occurring flavonoid compound, is found commonly in food, such as tea, onions, berries and apples. It exerts various beneficial effects through its anti-inflammatory and anti-oxidant properties. Quercetin intake is also correlated with lower incidence of coronary heart disease and stroke. Genistein, a potent phytoestrogen, is effective in mitigating endothelial dysfunction and exerts an anti-inflammatory effect by down-regulating the NFκB pathway. Plasma concentrations for genistein can range from 0.03-16.34 μM, in line with the dosage commonly used in in vitro studies. Quercetin has very low bioavailability in human plasma where the concentrations range between 0.3-3.5 μM. Nonetheless, higher concentrations of quercetin are known to be safe and well tolerated. Previous studies in human hepatocytes and human CRP mice have used quercetin at 10 μM. Therefore, the inventors chose to treat the stimulated hPSC-ECs with quercetin or genistein at 10 μM for up to 72 hours. However, gene expression of inflammatory markers did not show substantial reduction from the IL-1β stimulated levels at various time points (FIG. 3A). NFκB nuclear translocation levels remained elevated despite administration of quercetin and genistein (FIG. 3B). There was also no significant reduction of IL-8 protein levels from the conditioned media of stimulated hPSC-ECS after nutraceutical treatment for 48 hours (FIG. 3C).

The inventors further investigated whether the hPSC-ECs could metabolize the nutraceuticals. Using liquid chromatography-mass spectrometry (LC-MS) to analyze conditioned media from hPSC-ECs, the level of quercetin was found to decrease over time, while the levels of metabolites from both nutraceuticals did not increase remarkably (FIGS. 3D, 3E). Our data showed that firstly, the parent compounds may not be effective at eliciting anti-inflammatory effects on endothelial cells. Secondly, limited capacity of hPSC-ECs to break down the nutraceuticals into metabolites could have compromised the bioactivity of these compounds. Therefore, the inventors explored whether hepatocytes derived from hPSCs were capable of metabolizing the nutraceuticals.

### Example 5—HPSC-Derived Hepatocytes Bio-Activate Nutraceuticals Through Metabolism

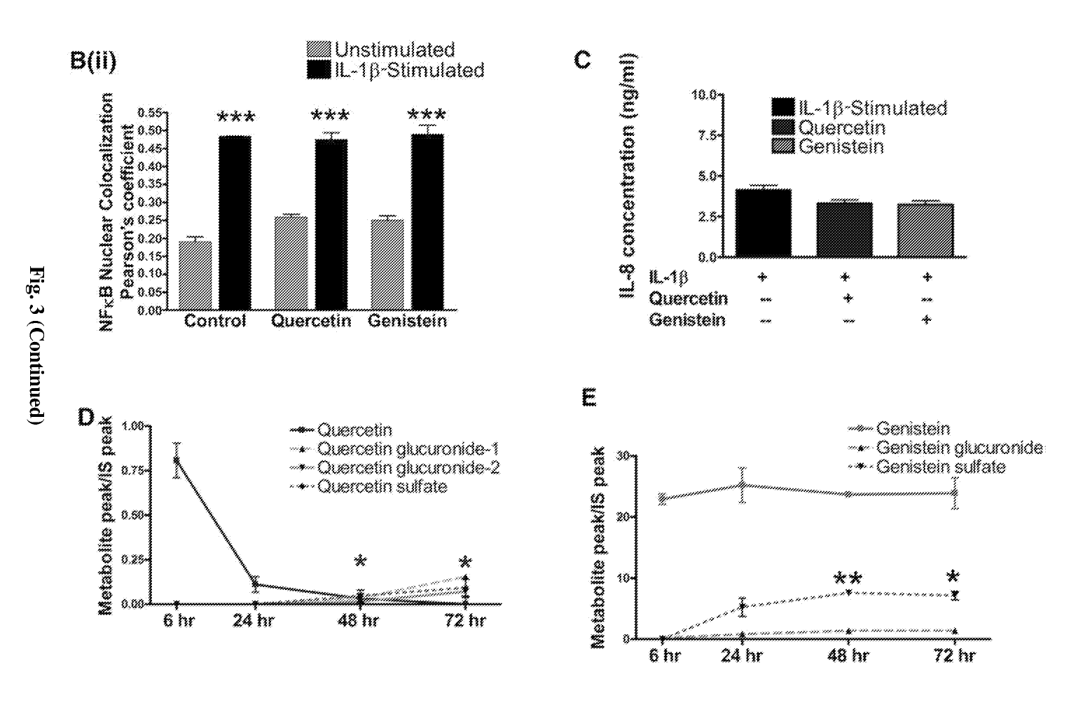

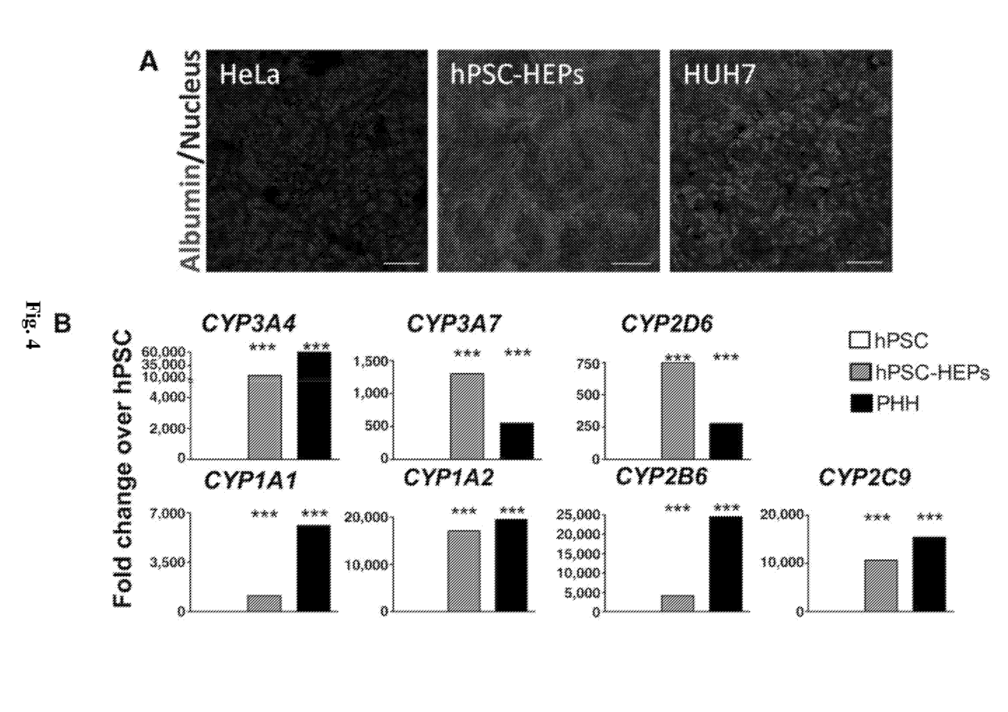

Liver has been shown to metabolize quercetin into its bioactive metabolites which in turn exert greater beneficial effects compared to their parent compound. The inventors generated hepatocytes from hPSCs following their established protocols [1, 3]. The stepwise differentiation protocol recapitulates embryonic liver development as hPSCs progressively turn from primitive streak/mesoendoderm, definitive endoderm and hepatoblasts to become hepatocytes (hereby referred to as hPSC-HEPs). Our hPSC-HEPs stained positive for albumin, characteristic of functional hepatocytes (FIG. 4A). The inventors produced hPSC-HEPs that expressed cytochrome P450 (CYP) genes (FIG. 4B), some of which were comparable to the positive control PHH. CYP enzymatic activities are necessary for metabolism of complex compounds.

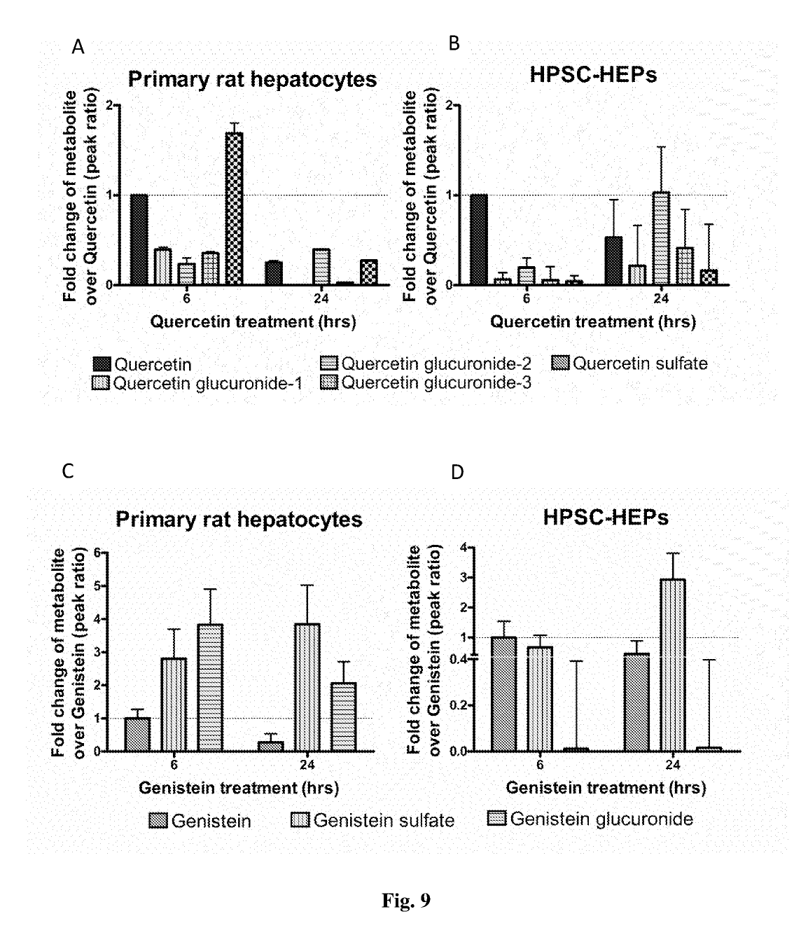

Indeed, LC-MS analysis demonstrated that the levels of both quercetin and genistein gradually declined over time in the presence of hPSC-HEPs (FIGS. 4C, 4D). Correspondingly, the levels of metabolites increased over time in the hPSC-HEPs. Most of the metabolites peaked at 48 hours and dropped by 72 hours. Thus, hPSC-HEPs were capable of converting quercetin and genistein into their metabolites, with 48 hours being the optimal duration based on the metabolic profiles of these nutraceuticals. When comparing the metabolic activity of hPSC-HEPs to freshly isolated primary rat hepatocytes, both demonstrated that quercetin and genistein declined over time, giving rise to metabolites (FIG. 9). As the primary rat hepatocytes could have retained some in vivo characteristics, their metabolic kinetics was apparently faster as substantial metabolites had emerged by 6 hours of treatment. The inventors then investigated the effects of nutraceuticals on IL-1β-stimulated hPSC-HEPs. A significant reduction in the panel of inflammatory gene expression upon nutraceutical treatment (FIG. 4E) was observed. The production of IL-8 protein from conditioned media of hPSC-HEPs was also significantly suppressed after treating with nutraceuticals for 48 hours (FIG. 4F). Hence, the ability of hPSC-HEPs to process nutraceuticals into their bio-active metabolites could have resulted in their efficacy in abrogating inflammatory activation.

### Example 6—Renewal of Nutraceutical Metabolites in the Presence of Hepatocytes Protects Endothelial Cells from Inflammatory Activation

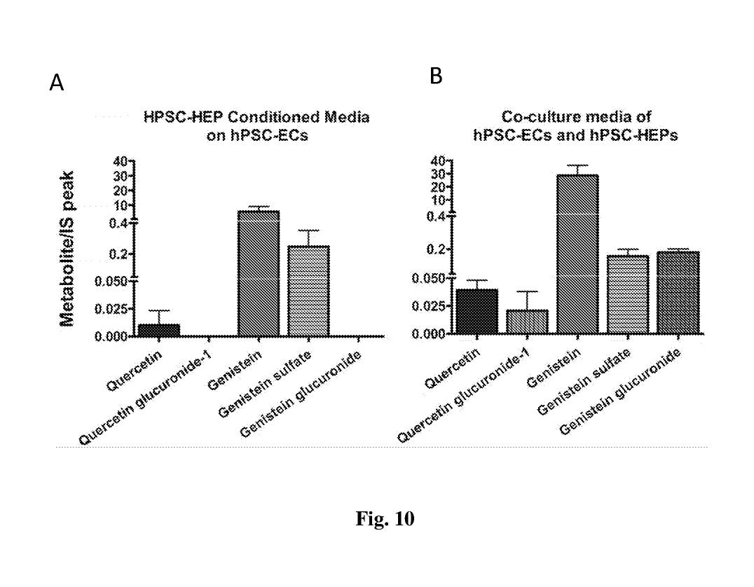

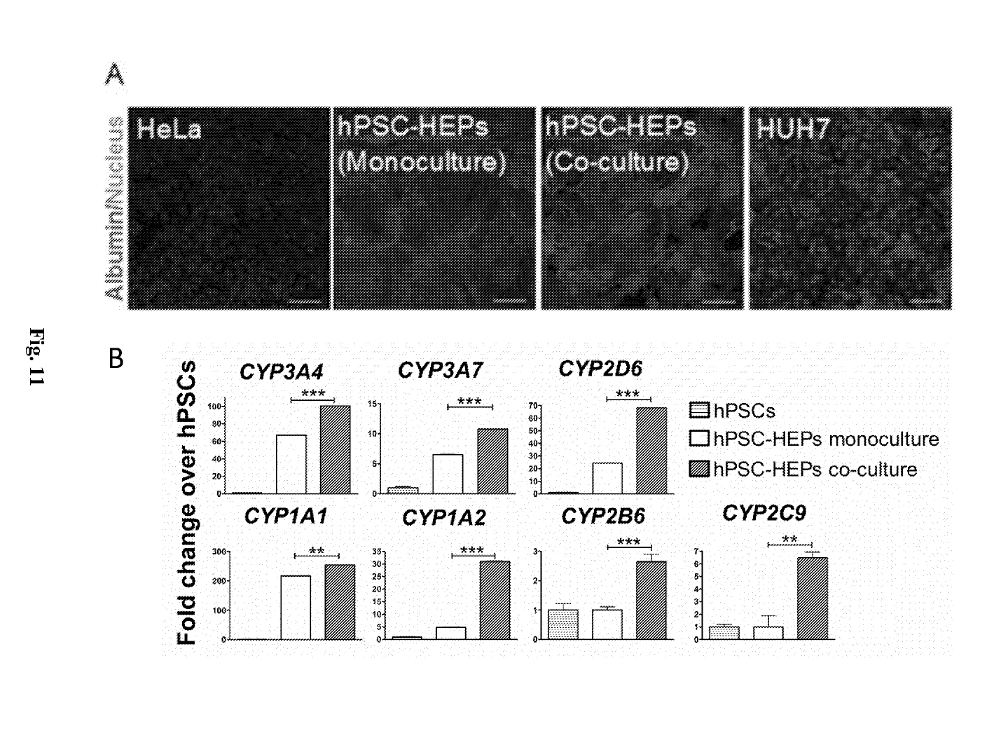

To enable accurate assessment of complex compounds on vascular health, two configurations of endothelial-hepatic paracrine interaction were examined. Firstly, 48-hour pre-incubation of each nutraceutical with hPSC-HEPs for metabolism was allowed to take place (FIG. 5A). Subsequently, hPSC-HEP conditioned media were collected and treated on hPSC-ECs under IL-1β stimulation. Alternatively, the inventors co-cultured hPSC-ECs and hPSC-HEPs on IBIDI μ-slide 2×9 wells where each cell type could be seeded separately into minor wells, and shared a common medium by filling up the major wells (FIG. 5A). The co-culture was pre-treated with each nutraceutical for 48 hours, followed by IL-1β stimulation. Our data showed that hPSC-HEP conditioned media with either quercetin or genistein did not seem to inhibit NFκB nuclear translocation in IL-1β-stimulated hPSC-ECs (FIG. 5B). In contrast, when co-cultured with hPSC-HEPs, stimulated hPSC-ECs displayed a significant suppression of NFκB nuclear translocation. Furthermore, the secretion of IL-8 in the co-culture setting was remarkably decreased, but not in the conditioned media configuration (FIG. 5C). The metabolite profiles of quercetin and genistein in each of the two configurations showed that there were detectable levels of various metabolites in the endothelial-hepatic co-culture (FIG. 10B) but not in the conditioned media setting (FIG. 10A). This supports that the metabolites in hPSC-HEP conditioned media could be degraded to a certain extent when they were subsequently treated on hPSC-ECs under IL-1β stimulation. This might lead to insufficient anti-inflammatory effects. On the other hand, the inventors also interrogated whether hPSC-ECs could in turn impact the metabolic function of hPSC-HEPs in a co-culture setting. The results show that co-cultured hPSC-HEPs had comparable albumin levels with mono-cultured hPSC-HEPs (FIG. 11A). Notably, there was significant increase of CYP gene expressions in co-cultured hPSC-HEPs (FIG. 11B), suggesting that the presence of hPSC-ECs could promote metabolic activity in hPSC-HEPs. Therefore, a co-culture of hPSC-ECs and hPSC-HEPs could better recapitulate the in vivo vascular-liver systemic interactome, with renewal of metabolites by liver metabolism.

In summary, the inventors have developed an endothelial-hepatic system to predict the efficacy of nutraceuticals in vascular protection. Insights from developmental studies guided our hPSC differentiation strategy. A protocol for efficient generation of functional endothelial cells from a lateral plate mesoderm precursor was established. Endothelial specification was induced by using FGF2, VEGF and 1% O₂, all of which play roles in blood vessel development and angiogenesis. Furthermore, small molecule SB431542, a potent antagonist of activin receptor-like kinase, could enhance the efficiency of endothelial differentiation by inhibiting TGF-β signalling which would otherwise promote mural cell specification from mesoderm. These hPSC-ECs were responsive to IL-1β-stimulated inflammation but treatment with either quercetin or genistein was not able to offset the inflammatory activation. This was due to limited metabolic activity of hPSC-ECs to break down the nutraceuticals into bioactive metabolites. Hence, it led the inventors to postulate that hepatocytes from hPSCs possess metabolic capacity to enhance bioavailability of metabolites from quercetin and genistein. Functional hepatocytes hPSC-HEPs with high expression of CYP enzymes were generated, which were able to effectively metabolize quercetin and genistein into primary and secondary metabolites. Similar metabolites were detected when comparing the nutraceutical treatment on hPSC-HEPs with primary rat hepatocytes, as well as those metabolites described in primary human hepatocytes. The inventors recognize that the primary rat hepatocytes required shorter time to metabolize the parent nutraceutical, as they were freshly isolated and hence could have retained most of their in vivo functionality. Depending on the structural complexities, it is also likely that different nutraceuticals would have distinct metabolic profiles. The hPSC-HEPs may require different treatment durations to release optimal level of metabolites. This also highlights the importance of dosage response in hPSC-HEPs, where a range of physiologically-relevant concentrations could be tested.

Notably, hPSC-HEP conditioned media containing nutraceutical metabolites were not effective in suppressing inflammation on hPSC-ECs. There could be a decline in the potency of metabolites from hPSC-HEP conditioned media due to degradation. Instead, when hPSC-ECs were co-cultured with hPSC-HEPs in a shared media, there was a significant reduction in inflammation. Continuous replenishment of metabolites in co-culture setup recapitulated the systemic setting of liver paracrine effects on the vasculatures. On the other hand, endothelial cells are known to improve hepatic function by promoting cell viability, synthesis of albumin and urea, and efficiency of drug transporter system. Our hPSC-ECs could in turn increase the metabolizing CYP enzyme activity in hPSC-HEPs. Another advantage of this endothelial-hepatic crosstalk is to recapitulate human-relevant response where certain drug metabolism dynamics may not be accurately reproduced in animals.

The endothelial-hepatic model disclosed herein could be capitalized for further development. Cells-on-chip in microfluidic-based systems could provide the benefits of different flow dynamics, minimizing reagents used, and optical suitability for high-content imaging of cells. Initial studies show that endothelial cells and hepatocytes have improved functionality in perfusion cultures. Phenotypic assays could also be developed to capture different pathological readouts for efficacy testing, as well as toxicology assessment. A spectrum of assay endpoints for vascular injury and atherosclerosis may include endothelial dysfunction, oxidative stress, apoptosis and matrix remodeling, etc. Multiplexing of phenotypic readouts in multicellular models would add great value to the applications of co-culture systems. In addition, our endothelial-hepatic platform could be utilized for disease modelling involving paracrine cross-talk between liver and vasculature. Since liver is integral to normal or dysfunctional lipid homeostasis, this interplay could influence vascular function. Moreover, it is likely that lipid-modifying nutraceuticals may involve liver metabolism to exert their actions on vascular tissue.

### REFERENCES

1. Tasnim, F., et al., Cost-effective differentiation of hepatocyte-like cells from human pluripotent stem cells using small molecules. Biomaterials, 2015. 70: p. 115-25.
2. Narmada, B. C., et al., HGF regulates the activation of TGF-beta1 in rat hepatocytes and hepatic stellate cells. Journal of cellular physiology, 2013. 228(2): p. 393-401.
3. Roelandt, P., et al., Human embryonic and rat adult stem cells with primitive endoderm-like phenotype can be fated to definitive endoderm, and finally hepatocyte-like cells. PloS one, 2010. 5(8): p. e12101.

1.-29. (canceled)

30. A method of deriving and maintaining endothelial cells, comprising:
(a) culturing lateral plate mesoderm cells under oxygen-deprived condition to obtain endothelial lineage cells; and
(b) culturing the endothelial lineage cells from (a) on an extracellular matrix to maintain and expand the endothelial lineage cells;
wherein the lateral plate mesoderm cells in (a) comprise splanchnic lateral plate mesoderm cells.

31. The method of claim 30, wherein the oxygen-deprived condition is carried out under oxygen supply of between 0.5% to 5%, or at about 1%.

32. The method of claim 30, wherein the lateral plate mesoderm cells are cultured in a first cell culture medium, wherein the first cell culture medium comprises:
(a) Fibroblast growth factor (FGF);
(b) TGF-β inhibitor; and
(c) Vascular endothelial growth factor (VEGF); optionally wherein the lateral plate mesoderm cells are cultured in the first cell culture medium for 3 to 7 days, or for about 5 days.

33. The method of claim 32, wherein the fibroblast growth factor is fibroblast growth factor 2 (FGF2); optionally wherein the fibroblast growth factor is at a concentration of between 1 to about 10 ng/ml, or at about 4 ng/ml.

34. The method of claim 32, wherein the TGF-β inhibitor is 4-[4-(1,3-benzodioxol-5-yl)-5-(2-pyridinyl)-1H-imidazol-2-yl]benzamide (SB 431542); optionally wherein the TGF-β inhibitor is at a concentration of between 1 to 30 μM, or at about 10 μM.

35. The method of claim 32, wherein the VEGF is VEGF-A; optionally wherein the VEGF-A is of an isoform of VEGF₁₆₅;
optionally wherein the VEGF is at a concentration of between 10 to 100 ng/ml, or at about 50 ng/ml.

36. The method of claim 32, wherein the fibroblast growth factor is fibroblast growth factor 2 (FGF2), the TGF-β inhibitor is 4-[4-(1,3-benzodioxol-5-yl)-5-(2-pyridinyl)-1H-imidazol-2-yl]benzamide (SB 431542), and the vascular endothelial growth factor is VEGF₁₆₅;
optionally wherein the FGF2 is at a concentration of between 1 to 10 ng/ml, or at about 4 ng/ml, wherein the SB 431542 is at a concentration of between 1 to 30 μM, or at about 10 μM, and wherein the VEGF₁₆₅ is at a concentration of between 10 to 100 ng/ml, or at about 50 ng/ml;
optionally wherein the FGF2 is at a concentration of 4 ng/ml, wherein the SB 431542 is at a concentration of 10 μM, and wherein the VEGF₁₆₅ is at a concentration of 50 ng/ml.

37. The method of claim 32, wherein the first culture medium does not comprise an activator of the Wnt signaling pathway.

38. The method of claim 30, wherein the endothelial lineage cells to be cultured on the extracellular matrix in (b) are characterized by the expression of one or more markers, wherein the one or more markers comprise at least one marker selected from the group consisting of PECAM-1, CD144 (VE-cadherin), Von Willebrand factor (vWF) and Endothelial NOS (eNOS) and combinations thereof; optionally wherein the endothelial lineage cells to be cultured on the extracellular matrix in (b) are characterized by the expression of PECAM-1, CD144 (VE-cadherin), Von Willebrand factor (vWF) and Endothelial NOS (eNOS).

39. The method of claim 30, wherein the lateral plate mesoderm cells to be cultured in the extracellular matrix in (b) are further characterized by the uptake of fluorescently-labelled acetylated low-density lipoprotein.

40. A cell co-culture system comprising an endothelial cell culture and a hepatocyte cell culture, wherein the endothelial cell culture is derived using a method of deriving and maintaining endothelial cells, comprising:
(a) culturing lateral plate mesoderm cells under oxygen-deprived condition to obtain endothelial lineage cells; and
(b) culturing the endothelial lineage cells from (a) on an extracellular matrix to maintain and expand the endothelial lineage cells;
wherein the lateral plate mesoderm cells in (a) comprise splanchnic lateral plate mesoderm cells.

41. The cell co-culture system of claim 40, wherein the hepatocyte cell culture comprises metabolically active hepatocyte cells.

42. The cell co-culture system of claim 40, wherein the endothelial cell culture and the hepatocyte cell culture are cultured in a co-culture medium, and wherein the co-culture medium comprises a mixture of an endothelial cell culture medium and a hepatocyte cell culture medium in the ratio of 2:1 to 1:2, or at about 1:1.

43. A method of disease modelling or drug testing using a cell co-culture system comprising an endothelial cell culture and a hepatocyte cell culture, wherein the endothelial cell culture is derived using a method of deriving and maintaining endothelial cells, comprising:
(a) culturing lateral plate mesoderm cells under oxygen-deprived condition to obtain endothelial lineage cells; and
(b) culturing the endothelial lineage cells from (a) on an extracellular matrix to maintain and expand the endothelial lineage cells;
wherein the lateral plate mesoderm cells in (a) comprise splanchnic lateral plate mesoderm cells.

44. The method of claim 43, wherein the disease modelling comprises modelling of inflammation or drug-induced vascular injury.

45. The method of claim 43, wherein the drug testing comprises testing the effect of the drug on the endothelial cell culture, testing drug-induced vascular injury, testing hepatotoxicity, predicting vascular protection, predicting drug efficacy or predicting drug safety.

46. The method of claim 45, wherein the testing of the effect of the drug on the endothelial cell culture drug testing comprises:
(a) co-culturing the endothelial cell culture and the hepatocyte cell culture with the drug to obtain hepatocyte metabolized drug; and
(b) measuring the effect of the hepatocyte metabolized drug from (a) on the endothelial cell culture.

47. The method of claim 46, wherein the endothelial cell culture and the hepatocyte cell culture are co-cultured with the drug for 24 hours to 3 days, or for about 48 hours.


SEQUENCE LISTING (38 sequences)

SEQ ID NO: 1 - DNA, 20 bp, Artificial Sequence
  CD31 forward primer
caggcgccgggagaagtgac

SEQ ID NO: 2 - DNA, 20 bp, Artificial Sequence
  CD31 reverse primer
cgtccagtccggcaggctct

SEQ ID NO: 3 - DNA, 21 bp, Artificial Sequence
  CDH5 forward primer
tggccagctggtcctgcagat

SEQ ID NO: 4 - DNA, 20 bp, Artificial Sequence
  CDH5 reverse primer
tgcccgtgcgacttggcatc

SEQ ID NO: 5 - DNA, 23 bp, Artificial Sequence
  TIE2 forward primer
gcagtgcaatgaagcatgccacc

SEQ ID NO: 6 - DNA, 20 bp, Artificial Sequence
  TIE2 reverse primer
ggtagcggccagccagaagc

SEQ ID NO: 7 - DNA, 21 bp, Artificial Sequence
  CD34 forward primer
cacaggagaaaggctgggcga

SEQ ID NO: 8 - DNA, 20 bp, Artificial Sequence
  CD34 reverse primer
tggccgtttctggaggtggc

SEQ ID NO: 9 - DNA, 20 bp, Artificial Sequence
  VWF forward primer
tagcccgcctccgccagaat

SEQ ID NO: 10 - DNA, 20 bp, Artificial Sequence
  VWF reverse primer
cctgcaggcgcaggtgaagt

SEQ ID NO: 11 - DNA, 22 bp, Artificial Sequence
  VEGFa forward primer
gcacataggagagatgagcttc

SEQ ID NO: 12 - DNA, 21 bp, Artificial Sequence
  VEGFa reverse primer
ccacagggacgggatttcttg

SEQ ID NO: 13 - DNA, 21 bp, Artificial Sequence
  NFKB1 forward primer
actacctggtgcctctagtga

SEQ ID NO: 14 - DNA, 23 bp, Artificial Sequence
  NFKB1 reverse primer
tttgacctgagggtaagacttct

SEQ ID NO: 15 - DNA, 21 bp, Artificial Sequence
  MCP1 forward primer
aagctcgcactctcgcctcca

SEQ ID NO: 16 - DNA, 24 bp, Artificial Sequence
  MCP1 reverse primer
gcattgattgcatctggctgagcg

SEQ ID NO: 17 - DNA, 22 bp, Artificial Sequence
  IL6 forward primer
aaattcggtacatcctcgacgg

SEQ ID NO: 18 - DNA, 23 bp, Artificial Sequence
  IL6 reverse primer
ggaaggttcaggttgttttctgc

SEQ ID NO: 19 - DNA, 25 bp, Artificial Sequence
  IL8 forward primer
ttggcagccttcctgatttctgcag

SEQ ID NO: 20 - DNA, 23 bp, Artificial Sequence
  IL8 reverse primer
acaaccctctgcacccagttttc

SEQ ID NO: 21 - DNA, 21 bp, Artificial Sequence
  VCAM1 forward primer
atggtcgcgatcttcggagcc

SEQ ID NO: 22 - DNA, 21 bp, Artificial Sequence
  VCAM1 reverse primer
aacggacttggccccctctgt

SEQ ID NO: 23 - DNA, 21 bp, Artificial Sequence
  ICAM1 forward primer
accggaaggtgtatgaactga

SEQ ID NO: 24 - DNA, 21 bp, Artificial Sequence
  ICAM1 reverse primer
tggttggctatcttcttgcac

SEQ ID NO: 25 - DNA, 23 bp, Artificial Sequence
  bFGF forward primer
agcgaccctcacatcaagctaca

SEQ ID NO: 26 - DNA, 23 bp, Artificial Sequence
  bFGF reverse primer
ctgcccagttcgtttcagtgcca

SEQ ID NO: 27 - DNA, 21 bp, Artificial Sequence
  CYP3A4  forward primer
aagtcgcctcgaagatacaca

SEQ ID NO: 28 - DNA, 21 bp, Artificial Sequence
  CYP3A4 reverse primer
aaggagagaacactgctcgtg

SEQ ID NO: 29 - DNA, 21 bp, Artificial Sequence
  CYP3A7 forward primer
tgctttgtccttccgtaaggg

SEQ ID NO: 30 - DNA, 21 bp, Artificial Sequence
  CYP3A7 reverse primer
cagcataggctgttgacagtc

SEQ ID NO: 31 - DNA, 21 bp, Artificial Sequence
  CYP2B6 forward primer
gcactcctcacaggactcttg

SEQ ID NO: 32 - DNA, 20 bp, Artificial Sequence
  CYP2B6 reverse primer
cccaggtgtaccgtgaagac

SEQ ID NO: 33 - DNA, 20 bp, Artificial Sequence
  CYP1A1 forward primer
acatgctgaccctgggaaag

SEQ ID NO: 34 - DNA, 20 bp, Artificial Sequence
  CYP1A1 reverse primer
ggtgtggagccaattcggat

SEQ ID NO: 35 - DNA, 21 bp, Artificial Sequence
  CYP1A2 forward primer
atgctcagcctcgtgaagaac

SEQ ID NO: 36 - DNA, 21 bp, Artificial Sequence
  CYP1A2 reverse primer
gttaggcaggtagcgaaggat

SEQ ID NO: 37 - DNA, 20 bp, Artificial Sequence
  CYP2C9 forward primer
gcctgaaacccatagtggtg

SEQ ID NO: 38 - DNA, 22 bp, Artificial Sequence
  CYP2C9 reverse primer
ggggctgctcaaaatcttgatg